(19) United States
(12) Patent Application Publication
(10) Pub. No.: US 2026/0256681 A1
(43) Pub. Date: Sep. 3, 2026

(54) HAIR CONDITIONING COMPOSITIONS CONTAINING A BLEND OF SILICONE AND TRIGLYCERIDES

(71) Applicant: The Procter & Gamble Company, Cincinnati, OH (US)

(72) Inventors: Jean Jianqun Zhao, Cincinnati, OH (US); MuiSiang Soh, Singapore (SG); Yu Wang, Singapore (SG); Zong Yi Chua, Singapore (SG); Jennifer Mary Marsh, Deerfield Township, OH (US)

(21) Appl. No.: 19/061,430

(22) Filed: Feb. 24, 2025

(51) Int. Cl. A61K 8/898; A61K 8/34; A61K 8/37; A61Q 5/02; A61Q 5/12

(52) U.S. Cl. CPC A61K 8/898; A61K 8/342; A61K 8/375; A61Q 5/02; A61Q 5/12; A61K 2800/592

(57) ABSTRACT

A hair conditioning composition includes a L-beta lamellar gel network, from about 0.1 wt. % to about 10 wt. % silicone, from about 0.1 wt. % to about 10 wt. % triglyceride, and a ratio of silicone to triglyceride from about 1:10 to about 10:1. The composition exhibits a shear stress from about 40 Pa to about 800 Pa @950 1/s. The L-beta lamellar gel network includes a surfactant, high melting point fatty compound, and an aqueous carrier. The surfactant may be a cationic surfactant. The hair conditioning composition may be a conditioner, a shampoo, or a 2-1 shampoo and conditioner.

### FIELD OF THE INVENTION

The present invention relates to hair conditioning compositions, more particularly to hair conditioning compositions comprising a blend of silicone and triglycerides.

### BACKGROUND OF THE INVENTION

A variety of approaches have been developed to condition the hair. These approaches range from post-shampoo application of hair conditioning such as leave-on and rinse-off products, to hair conditioning shampoos that attempt to both clean and condition the hair from a single product.

Although some consumers prefer the ease and convenience of a shampoo which includes conditioning, a substantial proportion of consumers prefer the more conventional conditioning formulations which are applied to the hair as a separate step from shampooing, usually after shampooing. Conditioning formulations can be in the form of rinse-off products or leave-on products, and can be in the form of an emulsion, cream, gel, spray, or mousse. Such consumers who prefer the conventional conditioning formulations may value the relatively higher conditioning effect, or the convenience of changing the amount of conditioning depending on the condition of hair or amount of hair.

Silicones are widely used in hair conditioning to provide a variety of hair benefits such as a reduction of combing force, improved slip feel, increased shine of hair, prevention of frizz, and retention of hair styles. However, there may be consumer drawbacks to at least some silicone materials. For example, some silicone materials may be difficult to wash off during shampooing, and, over time, may build up on the hair surface, making the hair heavy and weighing it down.

Natural oils and waxes have been formulated into hair conditioning in an attempt to replace silicone for hair conditioning. They are typically botanical triglyceride oils and waxes and may be oils such as coconut oil, capric caprylic triglycerides, shea butter, cocoa butter, pequi oil, argan oil, almond oil, apricot oil, rice bran oil, safflower oil, sunflower oil, hemp seed oil, avocado oil, grapeseed oil, evening primrose oil, camelia oil, moringa oil, meadowfoam seed oil, Crambe oil, castor oil, cottonseed oil, soybean oil, rapeseed oil, canola oil, candelilla wax, rice bran wax, sunflower wax, beeswax, bayberry wax, orange wax, and carnauba wax. The key consumer benefits of using natural oils in hair conditioning are hair moisturization and scalp health. However, there can be drawbacks, such as a draggy feel during the wet rinse, oils balling up on the dry hair surface resulting in an oily and greasy feel, and difficulty in creating and maintaining hair styles.

Thus, there is a need for hair conditioning that deliver the consumer acceptable benefits of silicone while minimizing the drawbacks of silicone and silicone alternatives.

### SUMMARY OF THE INVENTION

A. A hair conditioning composition comprising:
a L-beta lamellar gel network;
from about 0.1 wt. % to about 10 wt. % silicone;
from about 0.1 wt. % to about 10 wt. % triglyceride;
wherein the ratio of silicone to triglyceride is from about 1:10 to about 10:1;
wherein the composition has a shear stress from about 40 Pa to about 800 Pa @950 1/s according to the Shear Stress Method disclosed herein.

B. The hair conditioning composition of Paragraph A, wherein the L-beta lamellar gel network comprises surfactant, high melting point fatty compound, and an aqueous carrier.

C. The hair conditioning composition of Paragraph B, wherein the surfactant is a cationic surfactant, preferably wherein the cationic surfactant is selected from a mono-long alkyl amine; a di-long alkyl quaternized ammonium salt; mono-long alkyl cationic neutralized amino acid esters; a combination of a mono-long alkyl amine and a di-long alkyl quaternized ammonium salt; and a combination of a mono-long alkyl amine and a mono-long alkyl cationic neutralized amino acid esters; and combinations thereof.

D. The hair conditioning composition of any of the preceding Paragraphs A through C, wherein the high melting point fatty compound is selected from cetyl alcohol, stearyl alcohol, cetearyl alcohol, Brassica alcohol, behenyl alcohol, and combinations thereof.

E. The hair conditioning composition any of the preceding Paragraphs A through D, wherein the silicone is selected from polyalkyl siloxanes, polyaryl siloxanes, polyalkylaryl siloxanes, polyether siloxane copolymers, amino substituted silicones, alkylamino substituted silicones, quaternized silicones, aminosilicones, amodimethicones, terminal aminosilicones, branched aminosilicones, silicone polymers containing quaternary groups, and combinations thereof.

F. The hair conditioning composition of preceding Paragraphs A through E, wherein the silicone is an aminosilicone corresponding to the following formula:

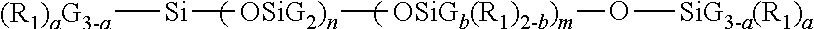

wherein G is selected from the group consisting hydrogen, phenyl, hydroxyl, C₁-C₈ alkyl, and methyl;
a is 0 or an integer having a value from 1 to 3, preferably 1;
b is 0, 1 or 2;
n is a number from 0 to 1,999;
m is an integer from 0 to 1,999; the sum of n and m is a number from 1 to 2,000; and wherein a and m are not both 0;
R₁ is a monovalent radical of formula CqH_{2q}L, wherein q is an integer from 2 to 8; and L is selected from the group consisting of:

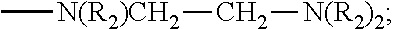

—N(R₂)₂;
—N(R₂)₃A⁻; and
—N(R₂)CH₂—CH₂—NR₂H₂A⁻;
wherein R₂ is selected from the group consisting of hydrogen, phenyl, benzyl, a saturated hydrocarbon radical, and an alkyl radical containing from about C₁ to about C_{20 atoms}; and A⁻ is a halide ion.

G. The hair conditioning composition of Paragraph F, wherein m is 0, a is 1, q is 3, G is methyl, n is from about 1,500 to about 1,700, and L is —N(CH) or —NH, preferably wherein m is 0, a is 1, q is 3, G is methyl, n is from about 400 to about 600, and L is —N(CH) or —NH.

H. The hair conditioning composition of Paragraph F or Paragraph G, wherein the aminosilicone comprises trimethylsilyamodimethicone.

I. The hair conditioning composition of any of Paragraphs F through H, wherein the aminosilicone has a viscosity of from about 1,000 centistokes to about 1,000,000 centistokes.

J. The hair conditioning composition of any of the preceding Paragraphs A through I, wherein the silicone is silicone polymer corresponding to the following formulas:

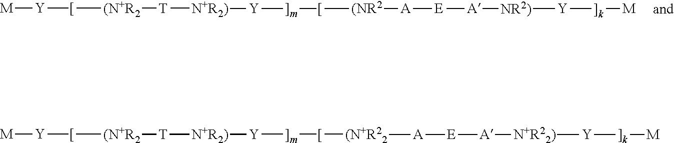

wherein:
m is an average value of from above 0 to 100
k is an average value of from above 0 to 50
M represents a terminal group, comprising terminal ester groups selected from
—OC(O)—Z;
—OS(O)₂—Z;
—OS(O₂)O—Z;
—OP(O)(O—Z)OH;
—OP(O)(O—Z)₂;
wherein Z is selected from monovalent organic residues having up to 40 carbon atoms,
wherein A and A′ each are independently selected from a single bond or a divalent organic group having up to 10 carbon atoms and one or more hetero atoms, and
E is a polyalkylene oxide group of the general formula:

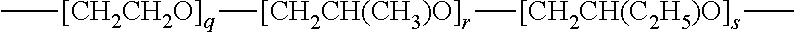

with
q=0 to 200,
r=0 to 200,
s=0 to 200,
and q+r+s=1 to 600,
R is selected from monovalent organic groups having up to 22 carbon atoms, and wherein the free valencies at the nitrogen atoms are bound to carbon atoms, R² is selected from hydrogen or R,
Y is a group of the formula:

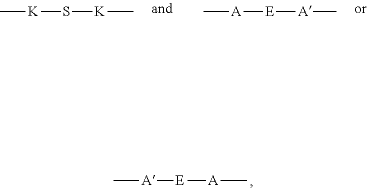

with

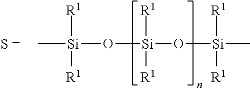

wherein R¹—C₁-C₂₂-alkyl, C₁-C₂₂-fluoroalkyl or aryl,
n=200 to 1000,
K is a bivalent or trivalent straight chain, cyclic and/or branched C₂-C₄₀ hydrocarbon residue,
wherein T is selected from a divalent organic group having up to 20 carbon atoms and one or more hetero atoms,
wherein the K residues in the —K—S—K— moiety are identical or different, and are bound to the silicon atom of the residue S via a C—Si— bond.

K. The hair conditioning composition of any of the preceding Paragraphs A through J, wherein the triglyceride is selected from rice bran oil, camellia oil, rapeseed oil, argon oil, avocado oil, soybean oil, safflower oil, canola oil, hemp seed oil, meadowfoam seed oil, coconut oil, palm kernel oil, caprylic/capric triglyceride, and combinations thereof.

L. The hair conditioning composition of any of the preceding Paragraphs A through K, wherein the conditioning composition is substantially free of an anionic surfactant.

M. The hair conditioning composition of any of the preceding Paragraphs A through L, wherein the L-beta lamellar gel network structure may comprise d-spacing of from about 5 nm to about 50 nm according to the Lamella Gel Network Test Method disclosed herein.

N. The hair conditioning composition of any of the preceding Paragraphs A through M, wherein the ratio of silicone to triglyceride is from about 8:1 to about 1:8, preferably about 5:1 to about 1:5.

O. The hair conditioning composition of any of the preceding Paragraphs A through N, wherein the pH of the composition is less than about 6, preferably less than about 5.

### BRIEF DESCRIPTION OF THE DRAWINGS

FIG. 1 is a chart illustrating the deposition of silicone on hair for Examples 1-5 and Comparative Examples 1-3.

FIG. 2 is a chart illustrating the deposition of triglyceride on hair for Examples 1-5 and Comparative Examples 4-5.

FIG. 3 is a chart illustrating the deposition of silicone on hair for Examples 6-10 and Comparative Example 7.

FIG. 4 is a chart illustrating the deposition of triglyceride on hair for Examples 6-10 and Comparative Examples 8-9.

### DETAILED DESCRIPTION OF THE INVENTION

Silicone has been used in hair conditioning compositions to provide hair benefits such as smooth hair feel, hair shine, hair moisturization, damaged hair repair, hair manageability, hair styling, and curl retention. However, silicones may have some potential drawbacks such as difficulty in washing off during shampooing, and, over time, build up on the hair surface, making the hair heavy and weighing it down. In the present invention, it has been surprisingly found that combining silicones with triglycerides results in a synergistic effect that enhances the deposition of both natural and silicone conditioning agents onto the hair. This results in improved overall conditioning performance over even the use of silicone alone, providing consumers with hair that may be softer, smoother, shinier, moisturized, and/or more manageable. Additionally, these conditioning compositions allow for a reduction in the total level of silicone needed, all while maintaining or even enhancing conditioning benefits to the hair.

All ingredient percentages described herein are by weight of the cosmetic composition, unless specifically stated otherwise, and may be designated as “wt %.” All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

“About” modifies a particular value by referring to a range of plus or minus 20% or less of the stated value (e.g., plus or minus 15% or less, 10% or less, or even 5% or less).

“Substantially free of” means a composition or ingredient comprises less than 3% of a subject material, by weight of the composition or ingredient (e.g., less than 2%, less than 1% or even less than 0.5%). “Free of” means a composition or ingredient contains 0% of a subject material.

Hair conditioning compositions may be used to improve the feel, appearance, and manageability of the hair. Hair conditioning compositions may comprise an L-beta lamellar gel network structure that is formed by the process of making (e.g., heating, emulsifying, and cooling) of compositions comprising (i) surfactant(s), (ii) high melting point fatty compound(s) having a melting point of greater than 25° C. and in some examples from 40° C. to 85° C., and (iii) an aqueous carrier. The L-beta lamellar gel network structure may provide: (a) consumer-desirable cosmetic appearance of creamy in-hand texture, slippery feel, and richness when spreading, (b) wet conditioning benefits, including improved wet detangling of the hair and slippery rinse feel, (c) dry hair protection benefits such as the repair of damaged hair and anti-statics, and (d) structure robustness to suspend and deliver hair conditioning active ingredients such as silicones, oils and particles.

In order to deliver such consumer benefits, the present inventive hair conditioning composition may include (a) a L-beta lamellar gel network; (b) from about 0.1 weight (“wt.”) % to about 10 wt. % of silicone; and (c) from about 0.1 wt. % to about 10 wt. % of triglyceride. The ratio of silicone to triglyceride may be from about 1:10 to about 10:1.

The hair conditioning composition may be in the form of a shampoo, a conditioner, or a multi-benefit composition that provides both shampooing and conditioning benefits to the hair in one product. The hair conditioning composition may be used as a leave-in or rinse-off product.

To deliver consumer benefits and to have the structure robustness of hair conditioning compositions, the L-beta lamellar gel network structure may comprise d-spacing of from about 5 nanometer (“nm”) to about 50 nm, as measured according to the d-spacing (L-beta spacing) of Lamella Gel Network Test Method. Also, the composition may have a shear stress from about 40 Pascal (“Pa”) to about 800 Pa @950 1/s.

The conditioning composition can have a shear stress from about 40 Pa to about 800 Pa, or from about 50 Pa to about 700 Pa, or from about 75 Pa to about 575 Pa, or from about 100 Pa to about 565 Pa, or from about 105 Pa to about 550 Pa, or from about 120 Pa, to about 500 Pa, or from about 125 Pa to about 450 Pa. The shear stress can be determined using the Shear Stress Method, described hereafter.

The conditioning composition can have a pH of less than about 6, or less than about 5, or from about 2.5 to about 5, alternatively from about 3.5 to about 4.5. The pH can be determined using the pH Test Method, described hereafter.

### L-Beta Lamellar Gel Network

The conditioning composition of the present invention may comprise a L-beta lamellar gel network. As used herein, the term “gel network” refers to a lamellar or vesicular solid crystalline phase which comprises at least one high melting point fatty compound, such as a fatty alcohol, as specified below, at least one surfactant, and water or other suitable solvents. The lamellar or vesicular phase comprises bi-layers made up of a first layer comprising the high melting point fatty compound and surfactant and alternating with a second layer comprising the water or other suitable solvent. Gel networks, generally, are further described by G. M. Eccleston, “Functions of Mixed Emulsifiers and Emulsifying Waxes in Dermatological Lotions and Creams”, Colloids and Surfaces A: Physiochem. and Eng. Aspects 123-124 (1997) 169-182; and by G. M Eccleston, “The Microstructure of Semisolid Creams”, Pharmacy International, Vol. 7, 63-70 (1986).

In a conditioning composition in the form of a conditioner, the L-beta lamellar gel network may be formed by combining (a) cationic surfactant; (b) a high melting point fatty compound; and (c) an aqueous carrier. In a conditioner, the composition may be substantially free of anionic surfactants, in view of stability of the gel network. In the present invention, such a conditioning composition may be “substantially free” of anionic surfactants, meaning that: the composition is free of anionic surfactants; or, if the composition contains anionic surfactants, the level of such anionic surfactants is very low. In such a conditioner composition, the total level of such anionic surfactants, if included, may be about 1 wt. % or less, alternatively about 0.5 wt. % or less, alternatively about 0.1 wt. % or less.

In a conditioning composition in the form of a shampoo, the L-beta lamellar gel network can be formed by combining (a) anionic surfactant, non-ionic surfactant, zwitterionic surfactant, or a combination thereof; (b) a high melting point fatty compound; and (c) an aqueous carrier.

When the L-beta lamellar gel network is formed, the surfactant and the high melting point fatty compound may be contained at a level such that the weight ratio of the surfactant to the high melting point fatty compound is in the range of from about 10:1 to about 1:10, alternatively from about 10:1 to about 1:7, alternatively from about 7:1 to about 1:10, alternatively from about 7:1 to about 1:7, in view of providing improved wet conditioning benefits.

### Surfactant

The L-beta lamellar gel network of the present invention includes surfactant. The surfactant may be included in the composition at a level of from about 0.1 wt. %, alternatively from about 0.5 wt. %, alternatively from about 0.8 wt. %, alternatively from about 1.0 wt. %, to about 30 wt. %, alternatively to about 20 wt. %, alternatively to about 15 wt. %, alternatively to about 12 wt. %, alternatively to about 10 wt. %, alternatively to about 8.0 wt. %, alternatively to about 6.0 wt. %.

The conditioning composition may have a total surfactant level of all surfactants in the composition of about 0.1 wt. % to about 30 wt. %, or about 0.1 wt. % to about 25 wt. %, or about 0.1 wt. % to about 20 wt. %, or about 0.1 wt. % to about 18 wt. %, or about 0.1 wt. % to about 16 wt. %, or about 0.4 wt. % to about 14 wt. %, or about 0.5 wt. % to about 12 wt. %, or about 0.5 wt. % to about 10 wt. %, or about 0.5 wt. % to about 8 wt. %, or about 0.5 wt. % to about 6 wt. %, or less than about 15 wt. %, or less than about 12 wt. %, or less than about 10 wt. %.

The surfactant can be water-insoluble. In the present invention, “water-insoluble surfactants” means that the surfactants have a solubility in water at 25° C. of alternatively below 0.5 g/100 g (excluding 0.5 g/100 g) water, alternatively 0.3 g/100 g water or less.

The surfactant may be one or more surfactants including ionic, zwitterionic, amphoteric, cationic, nonionic surfactants, or combinations thereof.

The surfactant in the L-beta lamellar gel network may include a cationic surfactant. For example, when the conditioning composition is in the form of a condition, the surfactant in the L-beta lamellar gel network may include a cationic surfactant.

The cationic surfactant may be one cationic surfactant or a combination of two or more cationic surfactants. The cationic surfactant may be a mono-long alkyl amine; a di-long alkyl quaternized ammonium salt; mono-long alkyl cationic neutralized amino acid esters; a combination of a mono-long alkyl amine and a di-long alkyl quaternized ammonium salt; and a combination of a mono-long alkyl amine and a mono-long alkyl cationic neutralized amino acid esters.

The surfactant may include amphoteric/zwitterionic surfactant such as those surfactants broadly described as derivatives of aliphatic secondary and tertiary amines in which one of the aliphatic substituents contains from 8 to 18 carbon atoms and one aliphatic substituent contains an anionic group such as a carboxy, sulfonate, phosphate, or phosphonate group; non-ionic surfactants such as the polyethylene oxide condensates of alkyl phenols; and combinations of these. Some non-limiting examples of the surfactants described above are disclosed in US 20190105246, US 20180098923, U.S. Pat. No. 9,271,908, WO 2020/016097, and McCutcheon's Emulsifiers and Detergents, 2019, MC Publishing Co.

Some suitable examples of surfactants include amphoteric surfactants selected from cocoamphoacetates, cocoamphodiacetates, lauroamphoacetates, lauroamphodiacetates, amidobetaines, amidosulfobetaines or combinations thereof.

Additional examples of amphoteric surfactants may include betaines, sultaines, hydroxysultanes, amphohydroxypropyl sulfonates, alkyl amphoactates, alkyl amphodiacetates, alkyl amphopropionates and combination thereof.

Examples of betaine amphoteric surfactants can include coco dimethyl carboxymethyl betaine, cocoamidopropyl betaine (CAPB), cocobetaine, lauryl amidopropyl betaine (LAPB), oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, cetyl betaine, or combinations thereof. Examples of sulfobetaines can include coco dimethyl sulfopropyl betaine, stearyl dimethyl sulfopropyl betaine, lauryl dimethyl sulfoethyl betaine, lauryl bis-(2-hydroxyethyl) sulfopropyl betaine or combinations thereof.

### Mono-Long Alkyl Amine—Cationic Surfactants

Mono-long alkyl amine cationic surfactants can include those having one long alkyl chain of alternatively from 19 to 30 carbon atoms, alternatively from 19 to 24 carbon atoms, alternatively from 20 to 24 carbon atoms, alternatively from 20 to 22 alkyl group. Mono-long alkyl amines can include mono-long alkyl amidoamines. Primary, secondary, and tertiary fatty amines can be used.

Tertiary amido amines may have an alkyl group of from about 19 to about 22 carbons. Exemplary tertiary amido amines include: behenamidopropyldimethylamine, behenamidopropyldiethylamine, behenamidoethyldiethylamine, behenamidoethyldimethylamine, brassicamidopropyldimethylamine, brassicamidopropyldiethylamine, brassicamidoethyldiethylamine, brassicamidoethyldimethylamine. Amines that may be used in the present invention are disclosed, for example, in U.S. Pat. No. 4,275,055, Nachtigal, et al.

The conditioning composition may be substantially free of or free of stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, arachidamidopropyldimethylamine, arachidamidopropyldiethylamine, arachidamidoethyldiethylamine, arachidamidoethyldimethylamine, and/or diethylaminoethylstearamide.

The amines described herein may be used in combination with acids such as L-glutamic acid, lactic acid, hydrochloric acid, malic acid, succinic acid, acetic acid, fumaric acid, tartaric acid, citric acid, L-glutamic hydrochloride, maleic acid, and combinations thereof; alternatively lactic acid, citric acid, at a molar ratio of the amine to the acid of from about 1:0.3 to about 1:2, alternatively from about 1:0.4 to about 1:1. The conditioning composition may contain from about 0.25 wt. % to about 6 wt. % acid, alternatively from about 0.4 wt. % to about 5 wt. % acid, from about 0.5 wt. % to about 4 wt. % acid, and alternatively from about 0.6 wt. % to about 3 wt. % acid.

The conditioning composition may be free of mono long alkyl quaternized ammonium salts.

### Mono-Long Alkyl Quaternized Ammonium Salt

The mono-long alkyl quaternized ammonium salts may have one long alkyl chain which has from 12 to 30 carbon atoms, or from 16 to 24 carbon atoms, or from C18-22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts may have the formula (A):

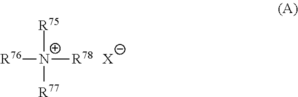

wherein one of R⁷⁵, R⁷⁶, R⁷⁷ and R⁷⁸ may be an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ may be independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ may be a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. One of R⁷⁵, R⁷⁶, R⁷⁷ and R⁷⁸ may be selected from an alkyl group of from 12 to 30 carbon atoms, from 16 to 24 carbon atoms, from 18 to 22 carbon atoms, or from 22 carbon atoms; the remainder of R⁷⁵, R⁷⁶, R⁷⁷ and R⁷⁸ may be independently selected from CH₃, C₂H₅, C₂H₄OH, and combinations thereof; and X may be selected from Cl, Br, CH₃OSO₃, C₂H₅OSO₃, and combinations thereof.

Nonlimiting examples of such mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

### Di-Long Alkyl Quaternized Ammonium Salts—Cationic Surfactants

When used, di-long alkyl quaternized ammonium salts may be combined with a mono-long alkyl quaternized ammonium salt and/or mono-long alkyl amine salt, at the weight ratio of from 1:1 to 1:5, alternatively from 1:1.2 to 1:5, alternatively from 1:1.5 to 1:4, in view of stability in rheology and conditioning benefits.

Di-long alkyl quaternized ammonium salts may have two long alkyl chains of from 12 to 30 carbon atoms, alternatively from 16 to 24 carbon atoms, alternatively from 18 to 22 carbon atoms. Such di-long alkyl quaternized ammonium salts may have the formula (B):

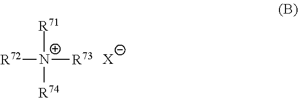

wherein two of R⁷¹, R⁷², R⁷³ and R⁷⁴ are selected from an aliphatic group of from 12 to 30 carbon atoms, alternatively from 16 to 24 carbon atoms, alternatively from 18 to 22 carbon atoms or an aromatic, alkoxy, polyoxyalkylene, alkylamido, hydroxyalkyl, aryl or alkylaryl group having up to about 30 carbon atoms; the remainder of R⁷¹, R⁷², R⁷³ and R⁷⁴ are independently selected from an aliphatic group of from 1 to about 8 carbon atoms, alternatively from 1 to 3 carbon atoms or an aromatic, alkoxy, polyoxyalkylene, alkylamido, hydroxyalkyl, aryl or alkylaryl group having up to about 8 carbon atoms; and X⁻ is a salt-forming anion selected from t halides such as chloride and bromide, C1-C4 alkyl sulfate such as methosulfate and ethosulfate, and combinations thereof. The aliphatic groups can contain, in addition to carbon and hydrogen atoms, ether linkages, and other groups such as amino groups. The longer chain aliphatic groups, e.g., those of about 16 carbons, or higher, may be saturated or unsaturated. Alternatively, two of R⁷¹, R⁷², R⁷³ and R⁷⁴ are selected from an alkyl group of from 12 to 30 carbon atoms, alternatively from 16 to 24 carbon atoms, alternatively from 18 to 22 carbon atoms; and the remainder of R⁷¹, R⁷², R⁷³ and R⁷⁴ are independently selected from CH₃, C₂H₅, C₂H₄OH, CH₂C₆H₅, and combinations thereof.

Di-long alkyl cationic surfactants may include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

### Alkyl Cationic Neutralized Amino Acid Esters—Cationic Surfactants

A neutralized amino acid ester that is a reaction product of a neutral amino acid having a non-polar side chain with a long chain fatty alcohol and is represented by formula (C):

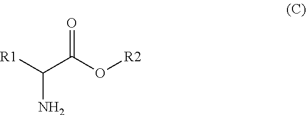

wherein R¹ is a linear or branched alkyl group; R² is a linear or branched carbon chain; and the amine group of the amino acid is neutralized with an acid. The present invention may comprise such materials, including those shown in U.S. Pat. Nos. 8,287,844 B2; 8,105,569 B2; and 11,207,249 B2, which are herein incorporated by reference.

An exemplary preferred neutralized amino acid ester may be Brassicyl L-isoleucine esylate (BLIE) or leucine isostearyl ester esylate (LIEE). Brassicyl L-isoleucine esylate (BLIE) may be derived from the esterification of Brassica alcohol with L-isoleucine esylate. L-isoleucine esylate may be prepared by reacting the amine group on isoleucine with ethanesulfonic acid. Brassica alcohol is a fatty alcohol that is derived from the splitting of high erucic acid rapeseed oil obtained from the Brassica genus of plants followed by hydrogenation. Brassica alcohol consists predominantly of stearyl (C₁₈), arachidyl (C₂₀) and behenyl (C₂₂) alcohols with minor quantities of lower and higher alkyl chain length alcohols. The conditioning compositions of the present invention may comprise a neutralized amino acid ester chosen from LIEE, BLIE or a combination thereof. Brassicyl valinate esylate (BVE) may be used.

### High Melting Point Fatty Compound

The composition of the present invention may comprise a high melting point fatty compound. The high melting point fatty compounds may be included in the composition at a level of from about 1.0 wt. % to about 30 wt. %, or from about 1.5 wt. % to about 20 wt. %, or from about 2.0 wt. % to about 15 wt. %, or from about 2.5 wt. % to about 8 wt. %.

The high melting point fatty compound may have a melting point of 25° C. or higher, alternatively 40° C. or higher, alternatively 45° C. or higher, alternatively 47° C. or higher, alternatively 49° C. or higher, in view of stability of the emulsion especially the gel network. Alternatively, such melting point may be up to about 90° C., alternatively up to about 80° C., alternatively up to about 75° C., even alternatively up to about 71° C., in view of easier manufacturing and easier emulsification.

In the present invention, the high melting point fatty compound can be used as a single compound or as a blend or combination of two or more high melting point fatty compounds. When used as such blend, the above melting point means the melting point of the blend.

The high melting point fatty compound may be selected from fatty alcohols, fatty acids, and combinations thereof. Further, it is understood by the artisan that, depending on the number and position of double bonds, and length and position of the branches, certain compounds having certain required carbon atoms may have a melting point of less than the above preferred in the present invention. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

If a fatty alcohol is used, the fatty alcohols may have from about 14 to about 30 carbon atoms, alternatively from about 16 to about 22 carbon atoms. These fatty alcohols may be saturated and can be straight or branched chain alcohols.

Fatty alcohols may include, for example, cetyl alcohol (having a melting point of about 56° C.), stearyl alcohol (having a melting point of about 58-59° C.), behenyl alcohol (having a melting point of about 71° C.), and combinations thereof. These compounds are known to have the above melting point. However, they often have lower melting points when supplied, since such supplied products are often combinations of fatty alcohols having alkyl chain length distribution in which the main alkyl chain is cetyl, stearyl, Brassica or behenyl group.

The fatty alcohol may be a combination of cetyl alcohol and stearyl alcohol. Generally, in the combination, the weight ratio of cetyl alcohol to stearyl alcohol is alternatively from about 1:9 to 9:1, alternatively from about 1:4 to about 4:1, alternatively from about 1:2.3 to about 1.5:1, alternatively from about 1:2 to about 1.2:1, alternatively from about 1:1.2 to about 1.2:1. When using higher level of total cationic surfactant and high melting point fatty compounds, the combination may have a weight ratio of cetyl alcohol to stearyl alcohol of from about 1:1 to about 4:1, or from about 1:1 to about 2:1, or from about 1.2:1 to about 2:1, in view of avoiding too thick for ease of spreadability. It may also provide more conditioning on damaged part of the hair.

### Aqueous Carrier

The composition of the present invention may include an aqueous carrier. The level and species of the carrier can be selected according to the compatibility with other components, and other desired characteristics of the product.

The carrier can include water and water solutions of lower alkyl alcohols. The lower alkyl alcohols can be monohydric alcohols having 1 to 6 carbons, such as ethanol and isopropanol.

The aqueous carrier may be substantially water. Deionized water may be used. Water from natural sources including mineral cations can also be used, depending on the desired characteristic of the product. The conditioning compositions of the present invention may comprise from about 40 wt. % to about 99 wt. %, alternatively from about 50 wt. % to about 95 wt. %, alternatively from about 70 wt. % to about 93 wt. %, alternatively from about 75 wt. % to about 95 wt. %, and alternatively from about 80 wt. % to about 92 wt. % aqueous carrier, alternatively at least about 60 wt. % aqueous carrier, alternatively at least about 70 wt. % aqueous carrier, alternatively at least about 80 wt. % aqueous carrier, alternatively at least 90 wt. % aqueous carrier.

### Silicone

The conditioning compositions of the present invention include a silicone or a blend of silicones. It is believed that silicone can provide smoothness and softness on dry hair. The conditioning compositions may include silicone at levels of from about 0.1 wt. % to about 10 wt. %, or from about 0.2 wt. % to about 9 wt. %, or from about 0.25 wt. % to about 8 wt. %, or from about 0.3 wt. % to about 7 wt. %, or from about 0.5 wt. % to about 6 wt. %, or from about 0.25 wt. % to about 5 wt. %, or from about 0.25 wt. % to about 4 wt. %.

The silicones may have an average particle size of from about 0.1 micron to about 200 microns, prefer 0.1 micron to 150 micron, prefer from 0.1 micron to 100 micron.

The silicones useful herein, as a single compound, as a blend or combination of at least two silicones, or as a blend or combination of at least one silicone and at least one solvent, may have a viscosity of from about 1,000 to about 2,000,000 mPa·s at 25° C. The viscosity can be measured by means of a glass capillary viscometer as set forth in Dow Corning Corporate Test Method CTM0004, Jul. 20, 1970.

Suitable silicone fluids include polyalkyl siloxanes, polyaryl siloxanes, polyalkylaryl siloxanes, polyether siloxane copolymers, amino substituted silicones, quaternized silicones, and combinations thereof. Other nonvolatile silicones having conditioning properties can also be used.

Polyalkyl siloxanes include, for example, polydimethylsiloxane, polydiethylsiloxane, and polymethylphenylsiloxane. Polydimethylsiloxane, which is also known as dimethicone, may be especially preferred.

The above polyalkylsiloxanes are available, for example, as a combination with silicones having a lower viscosity. Such combinations may have a viscosity of preferably from about 1,000 mPa·s to about 100,000 mPa·s, or from about 5,000 mPa·s to about 50,000 mPa·s. Such combinations may comprise: (i) a first silicone having a viscosity of from about 100,000 mPa·s to about 30,000,000 mPa·s at 25° C., preferably from about 100,000 mPa·s to about 20,000,000 mPa·s; and (ii) a second silicone having a viscosity of from about 5 mPa·s to about 10,000 mPa·s at 25° C., preferably from about 5 mPa·s to about 5,000 mPa·s. Such combinations useful herein include, for example, a blend of dimethicone having a viscosity of 18,000,000 mPa·s and dimethicone having a viscosity of 200 mPa·s available from GE Toshiba, and a blend of dimethicone having a viscosity of 18,000,000 mPa·s and cyclopentasiloxane available from GE Toshiba.

The silicones useful herein may include a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicones. This overlap is not intended as a limitation on any of these materials. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and combinations thereof. The silicone gums are available, for example, as a combination with silicones having a lower viscosity. Such combinations useful herein include, for example, Gum/Cyclomethicone blend available from Shin-Etsu.

### Aminosilicone

Silicones useful in the conditioning composition of present invention also include an aminosilicone, as provided herein, are silicones containing at least one primary amine, secondary amine, tertiary amine, or a quaternary ammonium group. Examples of preferred aminosilicones for use in embodiments of the subject invention include, but are not limited to, those which conform to the general formula (D):

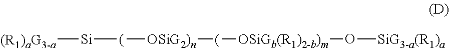

wherein G is hydrogen, phenyl, hydroxy, or C₁-C₈ alkyl, preferably methyl; a is 0 or an integer having a value from 1 to 3, preferably 1; b is 0, 1 or 2, preferably 1; n is a number from 0 to 1,999; m is an integer from 0 to 1,999; the sum of n and m is a number from 1 to 2,000; a and m are not both 0; R₁ is a monovalent radical conforming to the general formula CqH_{2q}L, wherein q is an integer having a value from 2 to 8 and L is selected from the following groups:

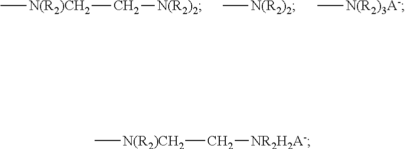

wherein R₂ is hydrogen, phenyl, benzyl, or a saturated hydrocarbon radical, preferably an alkyl radical from about C₁ to about C₂₀; A⁻ is a halide ion.

Preferred aminosilicones are those corresponding to formula (D) wherein m=0, a=1, q=3, G=methyl, n is preferably from about 1500 to about 1700, more preferably about 1600; and L is —N(CH₃)₂ or —NH₂, more preferably —NH₂. Other preferred aminosilicones are those corresponding to formula (D) wherein m=0, a=1, q=3, G=methyl, n is preferably from about 400 to about 600, more preferably about 500; and L is —N(CH₃)₂ or —NH₂, more preferably —NH₂. These aminosilicones can be called as terminal aminosilicones, as one or both ends of the silicone chain are terminated by nitrogen containing group.

An exemplary aminosilicone corresponding to formula (D) is the polymer known as “trimethylsilylamodimethicone”, which is shown below in formula (E):

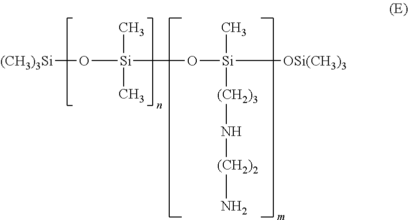

wherein n is a number from 1 to 1,999 and m is a number from 1 to 1,999.

In one of the preferred embodiments, the aminosilicone has a viscosity of from about 1,000 cs (centistokes) to about 1,000,000 cs, more preferably from about 10,000 cs to about 700,000 cs, more preferably from about 50,000 cs to about 500,000 cs, and still more preferably from about 100,000 cs to about 400,000 cs. This embodiment may also comprises a low viscosity fluid, such as, for example, those materials described below in Section F.(l). The viscosity of aminosilicones discussed herein is measured at 25° C.

In another preferred embodiment, the aminosilicone has a viscosity of from about 1,000 cs to about 100,000 cs, more preferably from about 2,000 cs to about 50,000 cs, more preferably from about 4,000 cs to about 40,000 cs, and still more preferably from about 6,000 cs to about 30,000 cs.

The aminosilicone is contained in the composition of the present invention at a level by weight of from about 0.05% to about 20%, preferably from about 0.1% to about 10%, and more preferably from about 0.3% to about 5%.

The above aminosilicones, when incorporated into the composition, can be mixed with solvent having a lower viscosity. Such solvents include, for example, polar or non-polar, volatile or non-volatile oils. Such oils include, for example, silicone oils, hydrocarbons, and esters. Among such a variety of solvents, preferred are those selected from non-polar, volatile hydrocarbons, volatile cyclic silicones, non-volatile linear silicones, and combinations thereof. The non-volatile linear silicones useful herein are those having a viscosity of from about 1 to about 20,000 centistokes, preferably from about 20 to about 10,000 centistokes at 25° C. The solvents may be non-polar, volatile hydrocarbons, especially non-polar, volatile isoparaffins, in view of reducing the viscosity of the aminosilicones and providing improved hair conditioning benefits such as reduced friction on dry hair. Such combinations may have a viscosity of from about 1,000 mPa·s to about 100,000 mPa·s, or from about 5,000 mPa·s to about 50,000 mPa·s. Other suitable alkylamino substituted silicones include those having alkylamino substitutions as pendant groups of a silicone backbone. Highly preferred are those known as “amodimethicone”. Commercially available amodimethicones useful herein include, for example, BY16-872 available from Dow Corning.

The aminosilicones useful herein may have non-quaternized amine functional groups such as primary, secondary, tertiary amine functional groups, and/or quaternary amine functional groups. The aminosilicones useful herein may be free of quaternary amine functional groups, thus, have non-quaternized amine functional groups such as primary, secondary, and/or tertiary amine functional groups.

The aminosilicones may have the amine functional groups: (i) as pendant groups attaching to a polysiloxane backbone; or (ii) at terminal ends of a polysiloxane backbone, or (i) aminosilicones having aminofunctional groups as pendant group attaching to a polysiloxane backbone.

The aminosilicone may be free of copolyol groups such as polyethylene glycol groups and polypropylene glycol groups, i.e., contain 0% of copolyol groups.

Such aminosilicones having non-quaternized aminofunctional groups as pendant group attaching to a polysiloxane backbone may include, but are not limited to silicones of the following formula F:

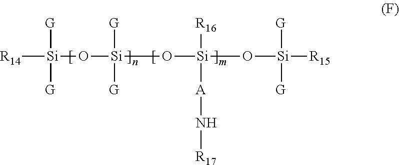

wherein:
a sum (n+m) ranges from about 2 to about 2000, preferably from about 150 to about 2000, more preferably from about 250 to about 1200, still more preferably from about 300 to about 800;
n is a number ranging from about 1 to about 1999, and m is a number ranging from about 1 to about 1999; and
n and m are chosen such that a ratio of m:n is from about 1:1000 to about 1:10, preferably from about 1:1000 to about 1:25, more preferably from about 1:800 to about 1:50, still more preferably from about 1:500 to about 1:50, even more preferably from about 1:400 to about 1:100;
R14, R15, R16, which may be identical or different, are chosen from a hydroxyl radical, C1-C4 alkoxy radicals and methyl, preferably R14 and R15 are hydroxyl radical and/or C1-C4 alkoxy radicals and R16 is methyl;
A is chosen from linear and branched C3-C8 alkenyl radicals;
R17 is chosen from H, phenyl, linear or branched C1-C4 alkyl radical, benzyl or preferably linear or branched (C2-C8)NH2; and
G is chosen from H, phenyl, hydroxyl, C1-C8 alkyl, preferably methyl.

These aminosilicones may be of the random or block type.

Suitable aminosilicones may include, but are not limited to, organomodified silicones with amine functionality available commercially under the trade names such as ADM1100 and ADM1600 from Wacker Silicones, AP6087, DC2-8211, DC8822, DC8822A, DC8803, DC2-8040, DC2-8813, DC2-8630 and DC8566 from Dow Corning Corporation, KF-862, KF-861, KF-8625, KF-8005, KF-8004, KF-8675, KF-873, and X-52-2328 from Shin-Etsu Corporation, and TSF 4702, TSF 4703, TSF 4704, TSF 4705, TSF 4707, TSF 4708, TSF 4709, F42-B3115, SF 1708, SF 1923, SF 1921, SF 1925, OF TP AC3309, OF 7747, OF-NH TP AI3631, OF-NH TP AI3683 from GE Bayer Silicones.

Aminosilicones of the present invention may include organomodified silicones with amine functionality with viscosities of greater than about 4,000 mPa·s in view of conditioning efficiency and up to about 100,000 mPa·s in view of friendly incorporation processing and spreadability, which include, but are not limited to, commercially available fluids under the trade names ADM1100 from Wacker Silicones, AP6087, DC8803 from Dow Corning Corporation, and TSF 4707 from GE Bayer Silicones.

The aminosilicone polymers which may be used in the compositions of the present invention may represented by the following formula G:

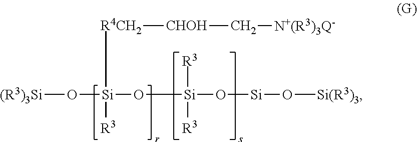

wherein R3 is a monovalent hydrocarbon radical from C1 to C18, preferably an alkyl or alkenyl radical, such as methyl; R4 is a hydrocarbon radical, preferably a C1 to C18 alkylene radical or a C10 to C18 alkyleneoxy radical, more preferably a C1 to C8 alkyleneoxy radical; Q- is a halide ion, preferably chloride; r is an average statistical value of from about 2 to about 20, preferably from about 2 to about 8; s is an average statistical value of from about 20 to about 200, preferably from about 20 to about 50, A preferred polymer of this class is known as UCARE SILICONE ALE 56™, available from Union Carbide.

The silicones may be incorporated in the present composition in the form of an emulsion, wherein the emulsion is made my mechanical mixing, or in the stage of synthesis through emulsion polymerization, with or without the aid of a surfactant selected from anionic surfactants, nonionic surfactants, cationic surfactants, and combinations thereof.

### Silicone Polymer Containing Quaternary Groups

Silicones useful herein include, for example, a Silicone Polymer Containing Quaternary Groups comprising terminal ester groups, having a viscosity up to 100,000 mPa·s and a D block length of greater than 200 D units. Without being bound by theory, this low viscosity silicone polymer provides acceptable conditioning benefits such as smooth feel, reduced friction, and prevention of hair damage, while eliminating the need for a silicone blend.

Structurally, the silicone polymer may be a polyorganosiloxane compound comprising one or more quaternary ammonium groups, at least one silicone block comprising greater than 200 siloxane units, at least one polyalkylene oxide structural unit, and at least one terminal ester group. The silicone block may comprise between 300 to 500 siloxane units.

The polyorganosiloxane compounds have the general formulas (H) and (I):

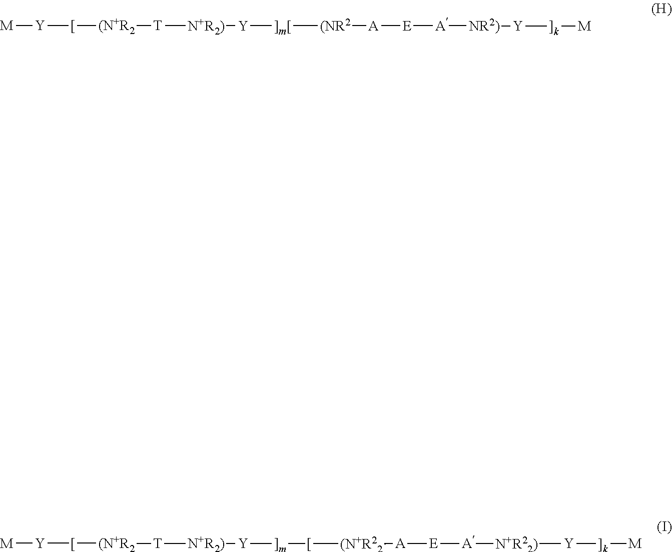

wherein:
m is >0, preferred 0.01 to 100, more preferred 0.1 to 100, even more preferred 1 to 100, specifically 1 to 50, more specifically 1 to 20, even more specifically 1 to 10,
k is 0 or an average value of from >0 to 50, or preferably from 1 to 20, or even more preferably from 1 to 10,
M represents a terminal group, comprising terminal ester groups selected from:

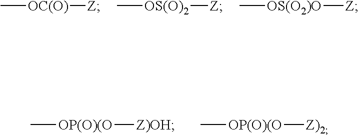

wherein Z is selected from monovalent organic residues having up to 40 carbon atoms, optionally comprising one or more hetero atoms.
A and A′ each are independently from each other selected from a single bond or a divalent organic group having up to 10 carbon atoms and one or more hetero atoms, and
E is a polyalkylene oxide group of the general formula:

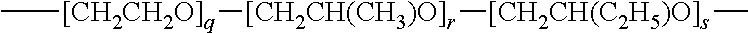

wherein q=0 to 200, r=O to 200, s=O to 200, and q+r+s=1 to 600.
R² is selected from hydrogen or R,
R is selected from monovalent organic groups having up to 22 carbon atoms and optionally one or more heteroatoms, and wherein the free valencies at the nitrogen atoms are bound to carbon atoms,
Y is a group of the formula:
—K—S—K— and -A-E-A′- or -A′-E-A-, with S=

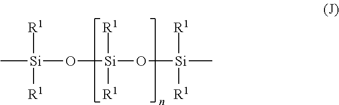

wherein R1=C₁-C₂₂-alkyl, C₁-C₂₂-fluoroalkyl or aryl; n=200 to 1000, and these can be identical or different if several S Groups are present in the polyorganosiloxane compound.
K is a bivalent or trivalent straight chain, cyclic and/or branched C₂-C₄₀ hydrocarbon residue which is optionally interrupted by —O—, —NH—, trivalent N, —NR¹—, —C(O)—, —C(S)—, and optionally substituted with —OH, wherein R¹ is defined as above,
T is selected from a divalent organic group having up to 20 carbon atoms and one or more hetero atoms.

The residues K may be identical or different from each other. In the —K—S—K— moiety, the residue K is bound to the silicon atom of the residue S via a C—Si-bond.

Due to the possible presence of amine groups (—(NR²-A-E-A′-NR²)—) in the polyorganosiloxane compounds, they may have protonated ammonium groups, resulting from the protonation of such amine groups with organic or inorganic acids. Such compounds are sometimes referred to as acid addition salts of the polyorganosiloxane compounds.

The molar ratio of the quaternary ammonium groups b) and the terminal ester groups c) may be less than 100:20, even more preferred is less than 100:30 and is most preferred less than 100:50. The ratio can be determined by ¹³C-NMR.

The polyorganosiloxane composition may comprise: A) at least one polyorganosiloxane compound, comprising a) at least one polyorganosiloxane group, b) at least one quaternary ammonium group, c) at least one terminal ester group, and d) at least one polyalkylene oxide group (as defined before), B) at least one polyorganosiloxane compound, comprising at least one terminal ester group, different from compound A).

In the definition of component A) it can be referred to the description of the polyorganosiloxane compounds. The polyorganosiloxane compound B) differs from the polyorganosiloxane compound A) preferably in that it does not comprise quaternary ammonium groups. Polyorganosiloxane compounds B) may result from the reaction of monofunctional organic acids, in particular carboxylic acids, and polyorganosiloxane containing bisepoxides.

In the polyorganosiloxane compositions the weight ratio of compound A) to compound B) may be less than 90:10. Or, in other words, the content of component B) is at least 10 weight percent. In the polyorganosiloxane compositions in compound A), the molar ratio of the quaternary ammonium groups b) and the terminal ester groups c) may be less than 100:10, less than 100:15 and less than 100:20.

The silicone polymer may have a viscosity at 20° C. and a shear rate of 0.1 s⁻¹ (plate-plate system, plate diameter 40 mm, gap width 0.5 mm) of less than 100,000 mPa·s (100 Pa·s). The viscosities of the neat silicone polymers may range from 500 to 100,000 mPa·s, or preferably from 500 to 70,000 mPa·s, or more preferably from 500 to 50,000 mPa·s, or even more preferably from 500 to 20,000 mPa·s. In further embodiments, the viscosities of the neat polymers may range from 500 to 10,000 mPa·s, or preferably 500 to 5000 mPa·s determined at 20° C. and a shear rate of 0.1 s⁻¹.

In addition to the above listed silicone polymers, the following compositions are provided below. For example, in the polyalkylene oxide group E of the general formula K:

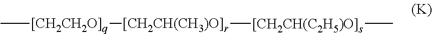

wherein the q, r, and s indices may be defined as follows:
q=0 to 200, or alternatively from 0 to 100, or alternatively from 0 to 50, or alternatively from 0 to 20,
r=0 to 200, or alternatively from 0 to 100, or alternatively from 0 to 50, or alternatively from 0 to 20,
s=0 to 200, or alternatively from 0 to 100, or alternatively from 0 to 50, or alternatively from 0 to 20, and
q+r+s=1 to 600, or alternatively from 1 to 100, or alternatively from 1 to 50, or alternatively from 1 to 40.

For polyorganosiloxane structural units with the general formula L:

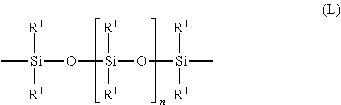

R¹═C₁-C₂₂-alkyl, C₁-C₂₂-fluoroalkyl or aryl; n=from 200 to 1000, or preferably from 300 to 500, K (in the group —K—S—K—) is preferably a bivalent or trivalent straight chain, cyclical or branched C₂-C₂₀ hydrocarbon residue which is optionally interrupted by —O—, —NH—, trivalent N, —NR¹—, —C(O)—, —C(S)—, and optionally substituted with —OH.

R¹ may be C₁-C₁₈ alkyl, C₁-C₁₈ fluoroalkyl and aryl. Furthermore, R¹ is preferably C₁-C₁₈ alkyl, C₁-C₆ fluoroalkyl and aryl. R¹ may be C₁-C₆ alkyl, C₁-C₆ fluoroalkyl, or C₁-C₄ fluoroalkyl, and phenyl. R¹ may be methyl, ethyl, trifluoropropyl and phenyl.

As used herein, the term “C₁-C₂₂ alkyl” means that the aliphatic hydrocarbon groups possess from 1 to 22 carbon atoms which can be straight chain or branched. Methyl, ethyl, propyl, n-butyl, pentyl, hexyl, heptyl, nonyl, decyl, undecyl, isopropyl, neopentyl and 1,2,3-trimethyl hexyl moieties serve as examples.

Further as used herein, the term “C₁-C₂₂ fluoroalkyl” means aliphatic hydrocarbon compounds with 1 to 22 carbon atoms which can be straight chain or branched and are substituted with at least one fluorine atom. Monofluoromethyl, monofluoroethyl, 1,1,1-trifluormethyl, perfluoroethyl, 1,1,1-trifluoropropyl, 1,2,2-trifluorobutyl are suitable examples.

Moreover, the term “aryl” means unsubstituted or phenyl substituted once or several times with OH, F, Cl, CF₃, C₁-C₆ alkyl, C₁-C₆ alkoxy, C₃-C₇ cycloalkyl, C₂-C₆ alkenyl or phenyl. Aryl may also mean naphthyl.

For the polyorganosiloxanes, the positive charges resulting from the ammonium group(s), may be neutralized with inorganic anions such as chloride, bromide, hydrogen sulfate, sulfate, or organic anions, like carboxylates deriving from C₁-C₃₀ carboxylic acids, for example acetate, propionate, octanoate, especially from C₁₀-C₁₈ carboxylic acids, for example decanoate, dodecanoate, tetradecanoate, hexadecanoate, octadecanoate and oleate, alkylpolyethercarboxylate, alkylsulphonate, arylsulphonate, alkylarylsulphonate, alkylsulphate, alkylpolyethersulphate, phosphates derived from phosphoric acid mono alkyl/aryl ester and phosphoric acid dialkyl/aryl ester. The properties of the polyorganosiloxane compounds can be, inter alia, modified based upon the selection of acids used.

The quaternary ammonium groups are usually generated by reacting the di-tertiary amines with an alkylating agents, selected from in particular di-epoxides (sometimes referred to also as bisepoxides) in the presence of mono carboxylic acids and difunctional dihalogen alkyl compounds.

The polyorganosiloxane compounds may be of the general formulas (M) and (N):

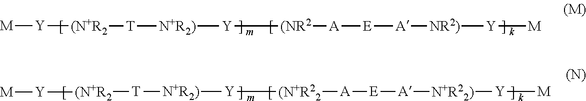

wherein each group is as defined above; however, the repeating units are in a statistical arrangement (i.e., not a block-wise arrangement).

The polyorganosiloxane compounds may be also of the general formulas (0) or (P):

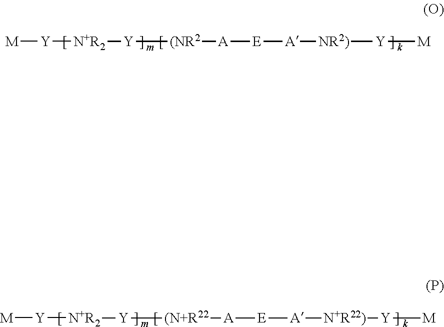

wherein each group is as defined above. Also in such formula the repeating units are usually in a statistical arrangement (i.e. not a block-wise arrangement).
wherein, as defined above, M is
—OC(O)—Z;
—OS(O)₂—Z;
—OS(O₂)O—Z;
—OP(O)(O—Z)OH;
—OP(O)(O—Z)₂;
Z is a straight chain, cyclic or branched saturated or unsaturated C₁-C₂₀, or preferably C₂ to C₁₈, or even more preferably a hydrocarbon radical, which can be interrupted by one or more —O—, or —C(O)— and substituted with —OH. M may be —OC(O)—Z resulting from normal carboxylic acids in particular with more than 10 carbon atoms like for example dodecanoic acid.

The molar ratio of the polyorganosiloxane-containing repeating group —K—S—K— and the polyalkylene repeating group -A-E-A′- or -A′-E-A- may between 100:1 and 1:100, or between 20:1 and 1:20, or between 10:1 and 1:10.

In the group —(N⁺R₂-T-N⁺R₂)—, R may represent a monovalent straight chain, cyclic or branched C₁-C₂₀ hydrocarbon radical, which can be interrupted by one or more —O—, —C(O)— and can be substituted by —OH, T may represent a divalent straight-chain, cyclic, or branched C₁-C₂₀ hydrocarbon radical, which can be interrupted by —O—, —C(O)— and can be substituted by hydroxyl.

The above described polyorganosiloxane compounds comprising quaternary ammonium functions and ester functions may also contain: 1) individual molecules which contain quaternary ammonium functions and no ester functions; 2) molecules which contain quaternary ammonium functions and ester functions; and 3) molecules which contain ester functions and no quaternary ammonium functions. While not limited to structure, the above described polyorganosiloxane compounds comprising quaternary ammonium functions and ester functions are to be understood as combinations of molecules comprising a certain averaged amount and ratio of both moieties.

Various monofunctional organic acids may be utilized to yield the esters. Exemplary compounds include C₁-C₃₀ carboxylic acids, for example C₂, C₃, C₈ acids, C₁₀-C₁₈ carboxylic acids, for example C₁₂, C₁₄, C₁₆ acids, saturated, unsaturated and hydroxyl functionalized Cis acids, alkylpolyethercarboxylic acids, alkylsulphonic acids, arylsulphonic acids, alkylarylsulphonic acids, alkylsulphuric acids, alkylpolyethersulphuric acids, phosphoric acid mono alkyl/aryl esters and phosphoric acid dialkyl/aryl esters.

### Triglyceride

The conditioning composition of the present invention further comprises a triglyceride or a blend of triglycerides. The triglyceride may be a natural, synthetic, or a blend of natural and synthetic oils.

The triglyceride may be present in the conditioning composition at a concentration from about 0.1 wt. % to about 10 wt. %. The weight ratio of silicone to triglyceride may be from about 1:10 to about 10:1, or about 1:8 to about 8:1, or about 5:1 to about 1:5.

The triglyceride may be a natural or synthetic oil of the following formula Q:

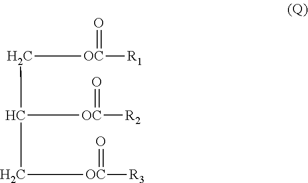

wherein R¹, R², and R³ are aliphatic hydrocarbyl groups that contain from about 2 to about 23 carbon atoms. The term “hydrocarbyl group” as used herein denotes a radical having a carbon atom directly attached to the remainder of the molecule. The aliphatic hydrocarbyl groups include the following:

(1) Aliphatic hydrocarbon groups; that is, alkyl groups such as heptyl, nonyl, undecyl, tridecyl, heptadecyl; alkenyl groups containing a single double bond such as heptenyl, nonenyl, undecenyl, tridecenyl, heptadecenyl, heneicosenyl; alkenyl groups containing 2 or 3 double bonds such as 8,11-heptadecadienyl and 8,11,14-heptadecatrienyl. All isomers of these are included, but straight chain groups are preferred.

(2) Substituted aliphatic hydrocarbon groups; that is groups containing non-hydrocarbon substituents which, in the context of this invention, do not alter the predominantly hydrocarbon character of the group. Those skilled in the art will be aware of suitable substituents; examples are hydroxy, carbalkoxy, (especially lower carbalkoxy) and alkoxy (especially lower alkoxy), the term, “lower” denoting groups containing not more than 7 carbon atoms.

(3) Hetero groups; that is, groups which, while having predominantly aliphatic hydrocarbon character within the context of this invention, contain atoms other than carbon present in a chain or ring otherwise composed of aliphatic carbon atoms. Suitable hetero atoms will be apparent to those skilled in the art and include, for example, oxygen, nitrogen and sulfur.

The triglycerides suitable for use in this invention may be vegetable oils and modified vegetable oils. The vegetable oil triglycerides are naturally occurring oils. By “naturally occurring” it is meant that the seeds from which the oils are obtained have not been subjected to any genetic altering. Further, by “naturally occurring” it is meant that the oils obtained are not subjected to hydrogenation or any chemical treatment that alters the di- and tri-unsaturation character. The naturally occurring vegetable oils that may be used include, for example, soybean oil, rapeseed oil, rapeseed oil containing oleic acid in a high concentration, corn oil, sesame oil, sesame salad oil, beafsteak plant oil, linseed oil, hemp oil, flaxseed oil, corn oil, peanut oil, safflower oil, safflower oil containing oleic acid in a high concentration, sunflower seed oil, sunflower seed oil containing oleic acid in a high concentration, cotton seed oil, grape seed oil, macadamia nut oil, hazel nut oil, pumpkinseed oil, walnut oil, camellia oil, tea seed oil, perilla oil, borage oil, olive oil, rice bran oil, wheat germ oil, palm oil, palm kernel oil, coconut oil, cacao butter, lesquerella oil, canola oil, meadowfoam oil, or castor oil.

The triglyceride oils may be modified vegetable oils. Triglyceride oils are modified either chemically or genetically. Hydrogenation of naturally occurring triglycerides is the primary means of chemical modification.

Triglycerides of the present invention may be short chain, medium chain, or long chain triglycerides or blends of short, medium, and/or long chain triglycerides. The triglyceride of the present invention may be a single triglyceride or a blend of triglycerides. Triglycerides comprised of fatty acid chains with from 2 to 5 carbon atoms are referred to as short chain triglycerides (“SCT”) and those with from 6 to 13 carbon atoms are referred to as medium chain triglycerides (“MCT”). Both SCT and MCT are invariably saturated and are found in dairy products as well as some plant oils. Those triglycerides comprised of fatty acid chains with 14 carbon atoms or greater are referred to as long chain triglycerides (“LCT”), may have points of unsaturation and are found in animal, fowl and fish products as well as plant oils.

SCT may be derived from specific fractions of unhydrogenated, partially hydrogenated or fully hydrogenated dairy butterfat, coconut oil, palm kernel oil and the like oils. Sources from which MCT and LCT can be derived are described further below. “Synthetic” triglycerides having the requisite short, medium and long chain fatty acid moieties may also be used in the present invention.

The triglycerides of the present invention are compounds having three molecules of the same or different acids esterified to glycerol (1,2,3-propanetriol) having the formula (CH2OH)2CHOH. The acids may be short, medium, and/or long such as the following exemplary formulas:

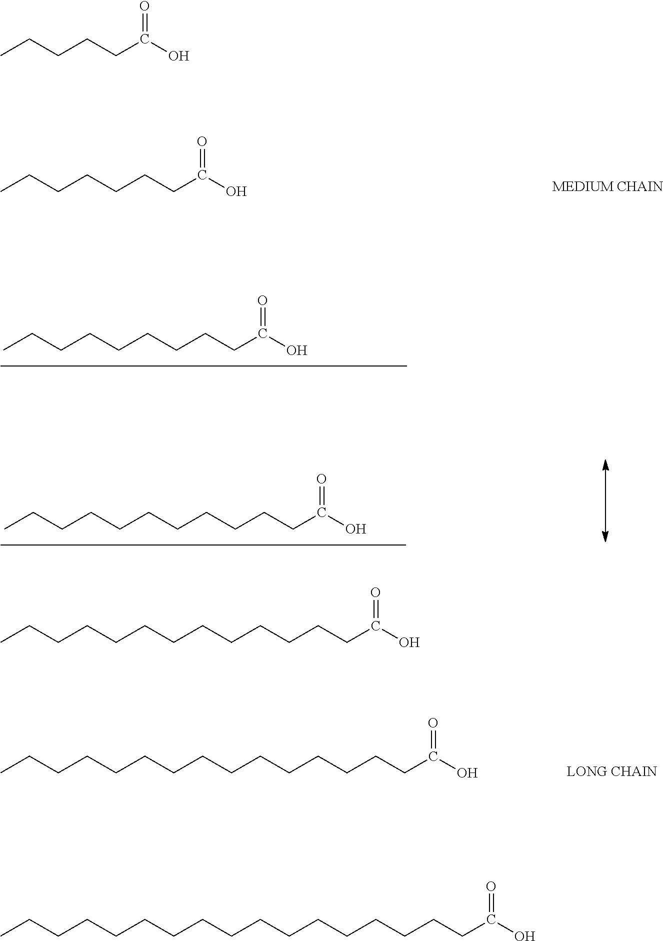

The short chain triglycerides may be either saturated or unsaturated, straight, or branched. The short chain triglycerides may be derived from any synthetic or natural organic acid, including, but not limited to butyric (butanoic), valeric (pentanoic), glycolic (hydroxyacetic), lactic (2-hydroxypropanoic), hydracrylic (3-hydroxypropanoic), hydroxybutyric, hydroxypentanoic and the like acids As used herein, chemical names include isomeric variations: for example, “butyric acid” includes normal butyric acid (butanoic) and iso-butyric (2-methylbutanoic acid), “valeric acid” includes normal valeric acid and iso-valeric (3-methylbutanoic) as so forth. The fatty acids may be butyric acid or combinations of these.

Combinations of short chain fatty acids may be derived from unhydrogenated, partially hydrogenated or fully hydrogenated dairy butterfat, palm kernel and the like oils.

The medium chain triglycerides may be those comprising from 6 to 13 carbon atoms, or from 6 to 10 carbon atoms or from 8 to 10 carbon atoms. They include, but are not limited to, C6 (caproic acid), C8 (caprylic acid), C10 (capric acid) and C12 (lauric acid) as well as combinations thereof. The medium chain fatty chain may comprise lipoic or thioctic acid in any one of its forms including alpha-lipoic acid.

Medium-chain triglycerides may be obtained by subjecting medium-chain fatty acids and glycerol to esterification reaction according to a conventional method, but there can preferably be used single acid triglycerides or mixed acid triglycerides composed of saturated fatty acids having 8 to 10 carbon atoms such as fatty acids obtained by hydrolysis of coconut oil, generally called MCT (Medium Chain Triglycerides), for example triglycerides of caprylic acid/capric acid=60/40^{~}75/25 (mass ratio).

The long chain triglycerides may be derived from any synthetic or natural, straight or branched, saturated or unsaturated, organic acid including, but no limited to palmitic (hexadecanoic), stearic (octadecanoic), arachidic (eicosanoic), behenic (docsanoic), lignoceric (tetracosanoic), cerotic (hexacosanoic), montanic (octacosanoic), melissic (triaconanoic) and the like acids. The LCT may also be derived by hydrogenating an unsaturated acid, including, but not limited to palmitoleic (9-hexadecenoic), oleic (cis 9-octadecenoic), elaidic (trans-9-octadecenoic), vaccenic (trans-11-octadecenoic), linoleic (cis, cis-9,12-octadecenoic), linolenic (9,12,15-octadecatrienoic and 6,9,12-octadecatrienoic), eleostearic (9,11,13-octadecatrienoic), arachidonic (5,8,11,14-eicosatetraenoic), nervonic (cis-15-tetracosenoic), eicosapentanoic, docosatetraenoic, docosapentaenoic, docosahexaenoic, and the like acids. Chemical names include isomeric variations.

The long chain triglycerides may be derived from, for example, non-hydrogenated, partially hydrogenated or fully hydrogenated oils such as soybean, safflower, sunflower, high oleic sunflower, sesame, peanut, corn, olive, rice bran, babassu nut, palm, mustard seed, cottonseed, poppyseed, low or high erucic rapeseed, shea, marine, meadowfoam, and the like oils. The long chain triglycerides may be derived from tallow, lard, shea butter, dairy butter, jojoba and combinations thereof.

The conditioning composition may include a blend of triglycerides. The blend of triglycerides may include SCT, MCT, and/or LCT. A non-limiting example of a blend of triglycerides may include LCT and MCT such as a blend of LCT of rice bran oil, argon oil, camellia oil, avocado oil, safflower oil, rapeseed oil, soybean oil, canola oil, hemp seed oil, meadowfoam seed oil, and MCT such as coconut oil, palm kernel oil, and caprylic/capric triglyceride.

The storage modulus (G′) represents how the cream stores energy before distortion, higher storage modulus would mean it can store more energy. G′ is relevant to the consumer feel of the product on the hand, before spreading. When compressed by the consumer, the conditioning composition feels thicker if the storage modulus is higher as it stores more energy. Conditioning compositions of the present invention may have a G′ of greater than about 1500 Pa, or greater than about 2000 Pa, or greater than about 2500 Pa.

Yield stress is measured as the oscillatory stress at which the linear viscoelastic region breakdowns. The yield stress is correlated with ease of spread felt by the consumer, as it is the minimum stress applied on the conditioning composition such that it starts to deform plastically. lower yield stress represents a lower amount of stress, and hence, a lower amount a force a consumer needs to spread the conditioning composition. Conditioning compositions of the present invention may have a yield stress of about 100 Pa or lower, or about 95 Pa or lower, or about 90 Pa or lower, or about 85 Pa or lower, about 80 Pa or lower. It has been surprising found that a conditioning composition comprising a blend of silicone with medium and long chain triglycerides provides a relatively high G′ and a relatively low yield stress, resulting in consumer satisfactory product feel and spread.

### Additional Components

The conditioning composition may include additional components such as conditioning agents, pH adjusters, sequestering agents, preservatives, perfumes, dyes, anti-dandruff agents, anti-fungals, and the like.

The conditioning composition may include conditioning agents such as aloe vera gel; aloe barbadensis leaf juice; ecklonia radiata extract; natural oils and waxes with shea butter, safflower oil, cocoa butter, orange peel wax, olive oil, macadamia seed oil, Oenothera biennis oil, Crambe abyssinica see oil, argon oil, camelina oil, sunflower oil, almond oil, Argania spinosa kernel oil, grape see oil, jojoba oil, coconut oil, meadowfoam seed oil, neem oil, linseed oil, castor oil, soybean oil, sesame oil, beeswax, sunflower wax, candelilla wax, rice bran wax, carnauba wax, bayberry wax and soy wax; essential oils such as lime peel oil, lavender oil, peppermint oil, cedarwood oil, tea tree oil, ylang-ylang oil and coensage oil which can be used in fragrance; hydrolyzed collagen with tradename Peptein 2000 available from Hormel, vitamin E with tradename Emix-d available from Eisai, panthenol available from Roche, panthenyl ethyl ether available from Roche, hydrolyzed keratin, proteins, plant extracts, and nutrients.

The conditioning composition may include pH adjusting agents, such as citric acid, sodium citrate, succinic acid, phosphoric acid, sodium hydroxide, sodium carbonate. The conditioning composition may include salts, such as potassium acetate and sodium chloride. The conditioning composition may include coloring agents, such as any of the FD&C or D&C dyes.

The conditioning composition may include sequestering agents, such as disodium ethylenediamine tetra-acetate; and ultraviolet and infrared screening and absorbing agents such as octyl salicylate; antioxidants include: rosemary, tocopherol, vitamin E, vitamin A, tea extracts, and hydroxyacetophenone (available as SymSave® H from Symrise®); amino acids include histidine, 1-arginine and others.

The conditioning composition can contain from about 0.2 wt. % to about 1.5 wt. % preservation system, alternatively from about 0.3 wt. % to about 1.25 wt. % preservation system, alternatively from about 0.4 wt. % to about 1 wt. % preservation system, alternatively from 0.5 wt. % to about 0.8 wt. % preservation system, and alternatively from about 0.6 wt. % to about 0.8 wt. % preservation system.

The conditioning composition can contain from about 0.05 wt. % to about 0.8 wt. % of a first preservation agent, such as sodium benzoate, alternatively 0.1 wt. % to about 0.5 wt. % sodium benzoate, alternatively from about 0.2 wt. % to about 0.4 wt. % sodium benzoate. The conditioning composition can contain sodium benzoate and can contain less than 2% sodium benzoate, alternatively less than 1.5% sodium benzoate, alternatively less than 1% sodium benzoate, alternatively less than 0.8% sodium benzoate, alternatively less than 0.6 wt. % sodium benzoate, and alternatively less than 0.5% sodium benzoate.

The preservation system may contain from about 20% to about 50% sodium benzoate, by weight of the preservation system, alternatively from about 25% to about 50% sodium benzoate, by weight of the preservation system, from about 30% to about 50% sodium benzoate, by weight of the preservation system, and from about 30% to about 40% sodium benzoate, by weight of the preservation system.

The conditioning composition may contain from about 0.3 wt. % to about 1.5 wt. % of a second preservation agent, such as a glycol and/or a glyceryl ester, alternatively from about 0.32 wt. % to about 1 wt. %, alternatively from about 0.33 wt. % to about 0.8 wt. %, alternatively from about 0.34 wt. % to about 0.6 wt. %, alternatively from about 0.35 wt. % to about 0.5 wt. %, alternatively from about 0.37 wt. % to about 0.45 wt. %, and alternatively from about 0.38 wt. % to about 0.43 wt. %. If the conditioning composition contains too much glycol and/or glyceryl esters the gel network structure may be destroyed, and the conditioning will not have consumer acceptable rheology and/or performance.

A preservation system may contain from about 50% to about 80% of the second preservation agent, by weight of the preservation system, alternatively from about 50% to about 75%, by weight of the preservation system, alternatively from about 50% to about 70%, by weight of the preservation system, and alternatively from about 50% to about 67%, by weight of the preservation system.

The weight ratio of sodium benzoate to the second preservation agent may be from about 1:4 to about 1:1, alternatively from about 1:3 to about 1:1, alternatively from about 1:2 to about 1:1, and from about 1:1.7 to about 1:1.

The conditioning compositions disclosed herein can comprise a perfume, which can be referred to as a perfume accord. The perfume can be suitable for application to the hair or skin.

The conditioning composition can contain from about 0.1 wt. % to about 5 wt. % perfume, alternatively from about 0.2 wt. % to about 3 wt. %, alternatively from about 0.3 wt. % to about 4 wt. %, alternatively from about 0.4 wt. % to about 2.5 wt. %, alternatively from about 0.5 wt. % to about 2 wt. %, alternatively from about 0.6 wt. % to about 1.5 wt. %, alternatively from about 0.6 wt. % to about 1.2 wt. %, and alternatively from about 0.7 wt. % to about 1 wt. % based on the total weight of the composition.

A wide variety of chemicals are known for fragrance (i.e., perfume) uses, including materials such as aldehydes, ketones and esters. More commonly, naturally occurring plant and animal oils and exudates comprising complex combinations of various chemical components are known for use as fragrances. The perfumes can be relatively simple in their compositions, comprising a single chemical, or can comprise highly sophisticated complex combinations of natural and synthetic chemical components, all chosen to provide any desired odor.

The perfume raw materials of the present compositions can have boiling points (BP) of about 500° C. or lower, alternatively about 400° C. or lower, alternatively about 350° C. or lower. The BP of many perfume raw materials are given in Perfume and Flavor Chemicals (Aroma Chemicals), Steffen Arctander (1969). The C log P value of the perfume raw materials useful herein can be greater than 0.1, alternatively greater than about 0.5, alternatively greater than about 1.0, alternatively greater than about 1.2.

The conditioning composition may include a soluble anti-dandruff agent. The soluble anti-dandruff agent may be one material or a combination selected from azoles, such as climbazole, ketoconazole, itraconazole, econazole, and elubiol; hydroxy pyridones, such as piroctone olamine, ciclopirox, rilopirox, and MEA-Hydroxyoctyloxypyridinone; kerolytic agents, such as salicylic acid and other hydroxy acids; strobilurins such as azoxystrobin and metal chelators such as 1,10-phenanthroline, and hinokitiol.

The azole anti-microbials may be an imidazole selected from benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and combinations thereof, or the azole anti-microbials is a triazole selected from terconazole, itraconazole, and combinations thereof. The azole anti-microbial agent may be ketoconazole. The sole anti-microbial agent may be ketoconazole.

The soluble anti-dandruff agent may be present in an amount from about 0.1% to 10%, in a further embodiment from about 0.25% to 8%, in yet a further embodiment from about 0.5% to 6%. Alternatively, the soluble anti-dandruff agent may be present in an amount of from about 0.1% to about 2%, alternatively from about 0.15% to about 1.5%, alternatively from about 0.2% to about 1%, alternatively from about 0.2% to about 0.75%, alternatively from about 0.25% to about 0.5%.

The conditioning composition may contain one or more particulate anti-dandruff agents. A safe and effective amount of anti-dandruff active for control of dandruff of the scalp is used. Particulate antidandruff agents include, for example, sulfur, selenium sulfide, and pyridinethione salts. Preferred are heavy metal salts of 1-hydroxy-2-pyridinethione and selenium disulfide. The particulate anti-dandruff agents are in crystalline form and are insoluble in the compositions. In general, particulate antidandruff agents can be present at levels of about 0.1% to about 5%, preferably from about 0.3% to about 2%, by weight of the composition. The particular amount used is not critical as long as a safe and effective amount is used for controlling dandruff when the composition is used to condition the hair.

The conditioning compositions of the present invention can be in the form of rinse-off products or leave-on products and can be formulated in a wide variety of product forms, including but not limited to creams, gels, emulsions, mousses, and sprays.

The conditioning compositions may be in the form of a shampoo, a conditioner, or a multi-benefit composition that provides both shampooing and conditioning benefits to the hair in one product.

The conditioning compositions of the present invention may be utilized as a system wherein the system includes a shampoo and a conditioner that both include a conditioning composition of the present invention.

The conditioning composition of the present invention may be in the form of a rinse-off hair conditioner. Such compositions are alternatively used by following steps:
(i) after shampooing hair, applying to the hair an effective amount of the conditioning compositions for conditioning the hair; and
(ii) then rinsing the hair.

The conditioning composition of the present invention may be in the form of a rinse-off shampoo. Such compositions are alternatively used by the following steps:
(i) apply to the hair an effective amount of the conditioning composition for shampooing the hair; and
(ii) optionally apply to the hair an effective amount of a conditioner for conditioning the hair; and
(iii) then rinsing the hair.

The conditioning composition of the present invention may be present in a 2-in-1 combined shampoo and conditioner product.

### Method of Making

The conditioning compositions described herein can be made using conventional methods.

### 1) L-Beta Lamellar Gel Network Formation:

In some aspects, the L-beta lamellar gel network may be made by: (a) combining a fatty alcohol, a surfactant, and an aqueous carrier at a temperature sufficient to allow partitioning of the surfactant and the aqueous carrier into the fatty alcohol to form a pre-mix; (b) cooling the pre-mix below the chain melt temperature of the fatty alcohol to form a gel network.

The L-beta lamellar gel network can be prepared by heating the fatty alcohol, the surfactant, and aqueous carrier to a level in the range of 75° C. to 90° C. and mixing. This mixture can be cooled to 27-35° C. (e.g., by passing the mixture through a heat exchanger). As a result of this cooling step, at least fifty percent of the mixture of the fatty alcohol and the surfactant crystallize to form a crystalline gel network.

The L-beta lamellar gel network can be prepared as following steps: a) preparing an oil phase comprising the surfactant and the fatty alcohol, wherein the temperature of the oil phase is from about 65° C. to about 90° C. which is higher than the melting point of the surfactant and the fatty alcohol, b) preparing an aqueous phase comprising the aqueous carrier, wherein the temperature is from about 25° C. to about 52° C. which is below the melting point of the surfactant and the fatty alcohol, c) mixing the oil phase and the aqueous phase to form gel network, where in the oil and water pre-mixes are injected in a Becomix® direct injection rotor-stator homogenizer to a high shear field having an energy density of from 1.0×10² J/m³ to 1.0×10⁷ J/m³.

Other methods of preparing the gel network phase include sonication and/or milling of the fatty alcohol, the surfactant, and aqueous carrier, while these components are heated, to reduce the particle size of the dispersed gel network phase. This results in an increase in surface area of the gel network phase, which allows the gel network surfactant and the aqueous carrier to swell the gel network phase. Another variation in preparing the gel network includes heating and mixing the fatty alcohol and the surfactant first, and then adding that mixture to the aqueous carrier.

2) Incorporating Conditioning Agents and Other Components into L-Beta Lamellar Gel Network:

The silicones, the triglycerides, the perfumes and other additional components are added into the gel network with constant stirring to assure homogenization. Then, the mixture is sent to a high shear milling homogenizer such as IKA DISPAX-REACTOR® DR 2000 at a rotor rotational speed from about 500 rpm to about 5000 rpm to suspend the silicones, the triglycerides, the perfumes and other components in gel network in preferred particle sizes, locations, and complex superstructures.

### Test Methods

### Differential Scanning Calorimetry Method

The melt transition behavior and temperature for the L-beta lamellar gel network may be obtained using differential scanning calorimetry (DSC) according to the following method. Utilizing a TA Instruments Q2000 DSC, approximately 15 mg of the L-beta lamellar gel network pre-mix or the final conditioning composition containing the gel network is placed into a Tzero aluminum hermetic DSC pan. The sample, along with an empty reference pan is placed into the instrument. The samples are analyzed using the following conditions/temperature program: Nitrogen Purge at a rate of 50.0 mL/min; Equilibrate @20.00° C.; Sampling interval 0.10 sec/pt; Equilibrate at 5.00° C.; Isothermal for 1.00 min; Ramp 5.00° C./min to 80.00° C. The resulting DSC data is analyzed using TA Instruments Universal Analysis Software.

The use of DSC to measure the melt transition behavior and temperature for L-beta lamellar gel networks is further described by T. de Vringer et al., Colloid and Polymer Science, vol. 265, 448-457 (1987); and H. M. Ribeiro et al., Intl. J. of Cosmetic Science, vol. 26, 47-59 (2004).

### pH Method

First, calibrate the Mettler Toledo Seven Compact pH meter. Do this by turning on the pH meter and waiting for 30 seconds. Then take the electrode out of the storage solution, rinse the electrode with distilled water, and carefully wipe the electrode with a scientific cleaning wipe, such as a Kimwipe®. Submerse the electrode in the pH 4 buffer and press the calibrate button. Wait until the pH icon stops flashing and press the calibrate button a second time. Rinse the electrode with distilled water and carefully wipe the electrode with a scientific cleaning wipe. Then submerse the electrode into the pH 7 buffer and press the calibrate button a second time. Wait until the pH icon stops flashing and press the calibrate button a third time. Rinse the electrode with distilled water and carefully wipe the electrode with a scientific cleaning wipe. Then submerse the electrode into the pH 10 buffer and press the calibrate button a third time. Wait until the pH icon stops flashing and press the measure button. Rinse the electrode with distilled water and carefully wipe with a scientific cleaning wipe.

Submerse the electrode into the testing sample and press the read button. Wait until the pH icon stops flashing and record the value.

Viscosity from Flow Curve Method

The viscosities of conditioning components or the final conditioning composition are measured by shear rate sweep condition with a rheometer available from TA Instruments with a mode name of DHR-3. The plate is called Peltier Plate. The temperature of the plate is kept at 25° C. Geometry has 40 mm diameter, cone angle of 2 degree, and gap of 55 μm. Shear rate ramp is between 0.1-1100 1/sec. Viscosities are reported at the shear rate of 2 s⁻¹ and 950 s⁻¹.

### Shear Stress Method

Shear stress is measured by shear rate sweep condition with a rheometer available from TA Instruments with a mode name of DHR-3. The plate is called Peltier Plate. The temperature of the plate is kept at 25° C. Geometry has 40 mm diameter, cone angle of 2 degree, and gap of 55 μm. Shear rate ramp is between 0.1-1100 1/sec. Shear stress at a high shear rate of 950 s⁻¹ is measured.

### Rheology Behavior Method (G′ Modulus and Yield Stress)

Yield stress is measured by oscillatory frequency sweep with a rheometer available from TA instruments with a mode name of DHR-2. The bottom plate is a sandblasted Peltier plate, and the top plate is a parallel sandblasted stainless-steel peltier plate. The temperature of the plate is kept at 25° C. Geometry has 40 mm diameter, and gap of 1000 um. Oscillatory amplitude sweep between 1-1000 Pa. Storage modulus (G′ modulus) is measured using the average storage modulus of the linear viscoelastic region.

### X-Ray Diffraction Method

SAXS (Small Angle X-ray Scattering) is used to confirm the presence of a multi-lamellar phase, and WAXS (Wide Angle X-ray Scattering) is used to differentiate between Lα (liquid) and Lβ (solid) crystalline structures were employed to verify the presence of the characteristic dispersed gel network phase of the personal conditioning compositions

### Lamella Gel Network Test Method

Small-angle x-ray scattering (“SAXS”) as used to resolve periodic structures in mesophases is essentially an x-ray diffraction technique. It is used in conjunction with conventional wide-angle x-ray scattering (“WAXS”) to characterize aggregate structures such as micelles, gel networks, lamella, hexagonal and cubic liquid crystals. The different mesophases that show periodic structures can be characterized by the relative positions (d-spacing) of their reflections as derived from the Bragg equation (d=λ/2 Sin θ) where d represents the interplanar spacing, λ the radiation wavelength and θ the scattering (diffraction) angle.

The one-dimensional lamella gel network phase is characterized by the ratio of the interplanar spacings d₁/d₁, d₁/d₂, d₁/d₃, d₁/d₄, d₁/d₅ having the values 1:2:3:4:5 etc. in the SAXS region (long-range order) and one or two invariant reflection(s) in the WAXS region (short-range) centered around 3.5 and 4.5 Å over a broad halo background. Other mesophases (e.g. hexagonal or cubic) will have characteristically different d-spacing ratios.

The SAXS data was collected with a Bruker NanoSTAR small-angle x-ray scattering instrument. The micro-focus Cu x-ray tube was operated at 50 kV, 0.60 mA with 550 um ScanTex Pinholes. The sample to detector distance was 107.39 cm and the detector a Vantec2K 2-dimensional area detector. Samples were sealed in capillaries and analyzed under vacuum with an analysis time of 600 s.

The value of d-spacing ((Lβ-beta spacing) of lamella gel network reported here is obtained with the 1^{st} order of SAXS reflection which is the d₁ spacing.

WAXS Confirmation (in Combination with SAXS) of Presence of Lβ Gel Network

Wide-angle data (WAXS) was collected on a Stoe STADI-MP diffractometer. The generator was operated at 40 kV/40 mA, powering a copper anode long-fine-focus Cu x-ray tube. The diffractometer incorporates an incident-beam curved germanium-crystal monochromator, standard incident-beam slit system, and Mythen PSD detector. Data were collected in transmission mode over a range of 0° to 50° 2θ with a step size of 3° 2θ and 15 seconds per step.

WAXS Pattern with reflection near 4.2 Å which, in combination with the lamellar reflections seen in the SAXS, is indicative of the presence of Lβ gel network.

### Optical Microscope Image Method

Conditioning compositions are examined under an Olympus BX61 Microscope using an Olympus DP72 camera (ISO 200, Exposure 3 sec) with lamp intensity 10V and air as refractive index (1.003). Microscope pictures were taken with objective lens of both 10× and 50×. Bright Field and Polarized Filter were used to examine the particle sizes of non-silicone hair conditioning agent compositions and gel network formation of conditioning compositions. Olympus cellSense was used as the software for imaging analysis.

### Deposition Method

Deposition of components of the conditioning compounds is measured by first treating hair samples according to the Hair Treatment Method disclosed herein. Following the Hair Treatment Method, the desired Deposition Method is then followed.

### 1) Hair Treatment Method:

a) Color treated Caucasian hair switches of 4 gram and 20.32 centimeters (“cm”) long (bound on root-ends with glue/tape and hanging on metal holder) are wet with 37.8 degrees Celcius water at a sink with a shower head fixture (flow rate is from 4.5 liters per minute to 5.7 liters per minute) for 30 seconds.
b) Hair shampoos (e.g. are applied at 0. L g of product per gram of hair (e.g., Herbal Essences Tea-Lightfully Clean Refreshing Shampoo or shampoo compositions of the present invention) via a syringe and milked/scrubbed for 30 seconds followed by a 30 seconds shower head rinse (with gentle manipulation at top of switch to ensure uniform rinsing).
c) Hair conditioners (Conditioner compositions of the present invention) are applied at a 0.1 grams of product per gram of hair via a syringe evenly over the hair switch and milked/scrubbed for 30 seconds followed by a 30 second shower rinse (with gentle manipulation at top of switch to ensure uniform rinsing).
d) The hair is then dried in a heat box set at 65.6 degrees Celsius or −45 minutes or until mostly dry.
e) For multiple cycle testing, the above procedure is repeated for a set number of times. For instance, for a three-cycle test, the above steps 1-4 are repeated three times.

For silicone deposition and triglyceride deposition, three cycle treated hair samples are used.

2) Silicone Deposition (ppm) Test Method:

For each sample from about 0.1 g to 0.2 g of hair was cut in 20-40 mm segments into vials. Then the hair was extracted gently with 2 ml extraction solvent which is a combination of methyl isobutyl ketone (MIBK) and Toluene (50/50 MIBK/Toluene). The vials are placed on a vortexer at 2 seconds on/1 second off, 2500 power for 30 minutes. The extracted solutions are filtered through a 13 mm 0.45 micron PTFE filter and run against an inorganic silicon calibration curve produced on an Optima 8300 ICP-OES system (Perkin Elmer, Waltham, Massachusetts) run in the axial mode. The silicon values determined are converted to a concentration of silicone polymer-equivalents deposited on the hair sample using the theoretical silicon concentration of the polymer provided by the manufacturer. An untreated hair sample is analyzed to determine the background concentration of silicon to allow correction if needed. Another untreated hair sample is spiked with a known amount of polymer and analyzed to ensure recovery of the polymer and verify the analysis.

3) Triglyceride Deposition (ppm) Test Method:

For each sample ~0.1 g of hair was cut in 20-40 mm segments into vials. Then the hair was extracted gently with hexane to remove the external triglyceride oil. The hexane extraction consists of extracting the hair with hexane two times (first with 10 ml then with 5 ml) with vortexing for 5 minutes each then discarding. An aliquot of this was then analyzed by GC. The triglycerides were analyzed directly without hydrolysis or derivatization. Separation and quantification of oil was measured by gas chromatography (GC) with flame ionization detection using a polydimethylsiloxane capillary column (2.5 m×0.250 mm×0.25 um) with hydrogen mobile phase at a 1.5 ml/ml flow rate with a 1 ul injection. The GC & FID was an Agilent 8890 type with the injector and detector at 350° C. The column oven was initially held at 150° C. then ramped at 20° C./min to 210° C. then at 35° C./min to 350° C. and help for 7 min. The injection was a split type with a split ratio of 50:1. Tridecanoin was used as an internal standard with a calibration curve for the triglycerides with a linear fit. For the more complex oils coconut oil was used to calibrate for the coconut oil and triolein for the other oils with all the triglyceride peaks quantitated. In addition, blank hair within a given experiment was analyzed and subtracted as background to determine only what additional triglyceride penetrated the hair.

### EXAMPLES

The following are non-limiting examples of the conditioning compositions described herein. It will be appreciated that other modifications of the present invention within the skill of those in the art can be undertaken without departing from the spirit and scope of this invention.

All parts, percentages, and ratios herein are by weight unless otherwise specified. Some components may come from suppliers as dilute solutions. The amount stated reflects the weight percent of the added material, unless otherwise specified.

TABLE 1
Inventive Examples 1-5 and Comparative Examples 1-5

| | | | | | | Comp. | Comp. | Comp. | Comp. | Comp. |
|---|---|---|---|---|---|---|---|---|---|---|
| | Ex. 1 | Ex. 2 | Ex. 3 | Ex. 4 | Ex. 5 | Ex. 1 | Ex. 2 | Ex. 3 | Ex. 4 | Ex. 5 |
| Si Deposition | 2510 | 1388 | 1122 | 471 | 369 | 1484 | 1366 | 301 | | |
| (mg/g) | | | | | | | | | | |
| TG (C16-C18) | 110 | 141 | 52 | 38 | 132 | | | | 82 | 33 |
| Deposition mg/g | | | | | | | | | | |
| Sheer stress (Pa) | 189 | 177 | 181 | 177 | 189 | 207 | 192 | 170 | 197 | 184 |
| @950 1/s | | | | | | | | | | |
| Behentrimonium | 4.664 | 4.664 | 4.664 | 4.664 | 4.664 | 4.664 | 4.664 | 4.664 | 4.664 | 4.664 |
| Methosulfate | | | | | | | | | | |
| (BTMS)¹ wt. % | | | | | | | | | | |
| (active) | | | | | | | | | | |
| Cetyl Alcohol | 0.297 | 0.297 | 0.297 | 0.297 | 0.297 | 0.297 | 0.297 | 0.297 | 0.297 | 0.297 |
| (C16 Fatty | | | | | | | | | | |
| alcohol) ³ | | | | | | | | | | |
| wt. % (active) | | | | | | | | | | |
| Stearyl Alcohol | 0.805 | 0.805 | 0.805 | 0.805 | 0.805 | 0.805 | 0.805 | 0.805 | 0.805 | 0.805 |
| (C18 Fatty | | | | | | | | | | |
| Alcohol) ⁴wt. % | | | | | | | | | | |
| (active) | | | | | | | | | | |
| Bis-Aminopropyl | 3 | 2 | 2 | 1 | 1 | 5 | 3 | 1 | | |
| Dimethicone | | | | | | | | | | |
| (TAS) ⁸ wt. % | | | | | | | | | | |
| Rice Bran Oil | 2 | 3 | 1 | 1 | 4 | | | | 5 | 3 |
| (RBO) wt. % | | | | | | | | | | |
| ratio of silicone | 3:2 | 2:3 | 2:1 | 1:1 | 1:4 | 5:0 | 3:0 | 1:0 | 0:5 | 0:3 |
| (TAS) to | | | | | | | | | | |
| triglyceride | | | | | | | | | | |
| (RBO) wt. % | | | | | | | | | | |
| Distilled Water | Q.S. | Q.S. | Q.S. | Q.S. | Q.S. | Q.S. | Q.S. | Q.S. | Q.S. | Q.S. |

Adjust pH w Citric acid to pH = 3.5-4.5

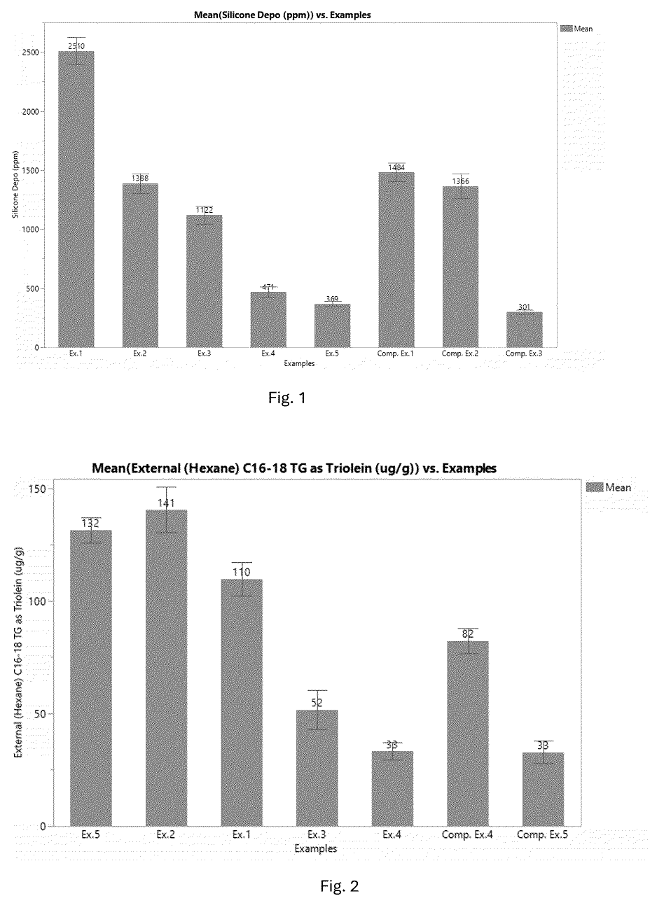

As shown in FIGS. 1 and 2 and as reported in Table 1, the conditioning compositions of Examples 1-5 having a combination of silicone and triglyceride demonstrate the improved silicone or triglyceride deposition on hair as compared to Comparative Examples 1-5 that contain silicone or triglyceride, but not in combination.

TABLE 2
Inventive Examples 6-10

| | Ex. 6 | Ex. 7 | Ex. 8 | Ex. 9 | Ex. 10 |
|---|---|---|---|---|---|
| Si Deposition (mg/g) | 1203 | 1088 | 925 | 625 | 1139 |
| TG (C08-C14 + C16-C18) Deposition mg/g | 84 | 84 | 227 | 137 | 105 |
| Microstructure (Clusters suspended in gel network “GN”) | yes | yes | yes | yes | yes |
| Sheer stress (Pa) @950 1/s | 333 | 357 | 346 | 352 | 180 |
| GN d-spacing (Å) | 308 | 310 | 308 | 314 | 322 |
| Behentrimonium Methosulfate (BTMS) ¹ wt. % (active) | 2.376 | 2.376 | 2.376 | 2.376 | 2.376 |
| Cetyl Alcohol (C16 Fatty alcohol) ³ wt. % (active) | 1.12 | 1.12 | 1.12 | 1.12 | 1.12 |
| Stearyl Alcohol (C18 Fatty Alcohol) ⁴ wt. % (active) | 2.862 | 2.862 | 2.862 | 2.862 | 2.862 |
| DiSodium EDTA wt. % (active) | 0.127 | 0.127 | 0.127 | 0.127 | 0.127 |
| Benzyl Alcohol wt. % (active) | 0.4 | 0.4 | 0.4 | 0.4 | 0.4 |
| Perfume wt. % | 0.8 | 0.8 | 0.8 | 0.8 | 0.8 |
| Methylchloroisothiazolinone/Methyl ⁷ wt. % | 0.033 | 0.033 | 0.033 | 0.033 | 0.033 |
| Bis-Aminopropyl Dimethicone (TAS) ⁸ wt. % | 3 | 3 | 3 | 2 | 2 |
| Dimethicone (PDMS) ⁹wt. % | | | | | 0.8 |
| PEG/PPG-20/23 Dimethicone (SS) ¹⁰wt. % | | | | | 0.2 |
| Rice Bran Oil (RBO) wt. % | 2 | 1.5 | | 2 | 2 |
| Caprylic/Capric Triglyceride (CCT) wt. % | | 0.5 | 2 | 0.5 | |
| total silicone (TAS + PDMS + SS) wt. % | 3 | 3 | 3 | 2 | 3 |
| total triglyceride (RBO + CCT) wt. % | 2 | 2 | 2 | 2.5 | 2 |
| ratio of silicone to triglyceride (RBO) | 3:2 | 3:2 | 3:2 | 4:5 | 3:2 |
| Distilled Water | Q.S. | Q.S. | Q.S. | Q.S. | Q.S. |

Adjust pH w Citric acid to pH = 3.5-4.5

TABLE 3
Comparative Examples 6-9

| | Comp. | Comp. | Comp. | Comp. |
|---|---|---|---|---|
| | Ex. 6 | Ex. 7 | Ex. 8 | Ex. 9 |
| Si Deposition (mg/g) | 353 | 837 | | |
| TG (C08-C14 + C16-C18) Deposition mg/g | | | 29 | 263 |
| Microstructure (Clusters suspended in GN) | no | no | no | no |
| Sheer stress (Pa) @950 1/s | 205 | 308 | 341 | 335 |
| GN d-spacing (Å) | 306 | 320 | 306 | 306 |
| Behentrimonium Methosulfate (BTMS) ¹ wt. % | 2.376 | 2.376 | 2.376 | 2.376 |
| (active) | | | | |
| Cetyl Alcohol (C16 Fatty alcohol) ³ wt. % (active) | 1.12 | 1.12 | 1.12 | 1.12 |
| Stearyl Alcohol (C18 Fatty Alcohol) ⁴ wt. % (active) | 2.862 | 2.862 | 2.862 | 2.862 |
| DiSodium EDTA wt. % (active) | 0.127 | 0.127 | 0.127 | 0.127 |
| Benzyl Alcohol wt. % (active) | 0.4 | 0.4 | 0.4 | 0.4 |
| Perfume wt. % | 0.9 | 0.8 | 0.8 | 0.8 |
| Methylchloroisothiazolinone/Methyl ⁷ wt. % | 0.033 | 0.033 | 0.033 | 0.033 |
| Bis-Aminopropyl Dimethicone (TAS) ⁸ wt. % | 2.1 | 3 | | |
| Dimethicone (PDMS) ⁹wt. % | 2 | | | |
| PEG/PPG-20/23 Dimethicone (SS) ¹⁰wt. % | 0.5 | | | |
| Rice Bran Oil (RBO) wt. % | | | 3 | 3 |
| Caprylic/Capric Triglyceride (CCT) wt. % | | | | 1.5 |
| total silicone (TAS + PDMS + SS) wt. % | 4.6 | 3 | | |
| total triglyceride (RBO + CCT) wt. % | | | 3 | 4.5 |
| ratio of silicone to triglyceride (RBO) | 4.6:0 | 3:0 | 0:3 | 0:4.5 |
| Distilled Water | Q.S. | Q.S. | Q.S. | Q.S. |

Adjust pH w Citric acid to pH = 3.5-4.5

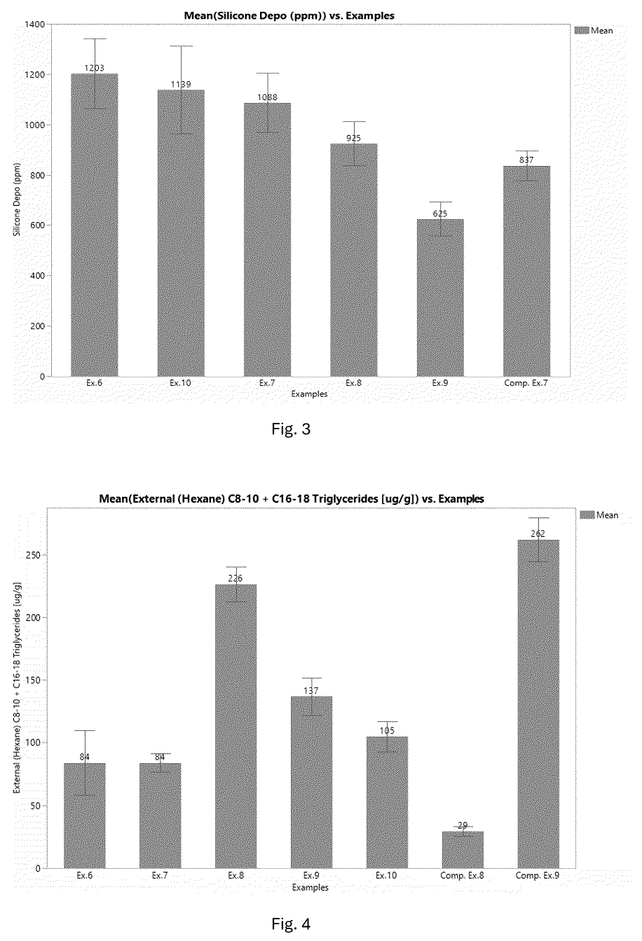

As shown in FIGS. 3 and 4 and as reported in Tables 2 and 3, the conditioning compositions of Examples 6-10 having a combination of silicone and triglyceride demonstrate the improved silicone or triglyceride deposition on hair as compared to Comparative Examples 6-9 that contain silicone or triglyceride, but not in combination.

TABLE 4
Inventive Examples 11-17

| | Ex. 11 | Ex. 12 | Ex. 13 | Ex. 14 | Ex. 15 | Ex. 16 | Ex. 17 |
|---|---|---|---|---|---|---|---|
| Sheer stress (Pa) | 318 | 328 | 316 | 335 | 330 | 361 | 370 |
| @950 1/s | | | | | | | |
| GN d-spacing (nm) | 229 | 220 | 225 | 222 | 225 | 220 | 310 |
| Stearamidopropyl | 1.95 | 1.95 | 1.95 | 1.95 | 1.95 | 1.95 | |
| Dimethylamine | | | | | | | |
| (SAPDMA)² | | | | | | | |
| wt. % | | | | | | | |
| Behentrimonium | | | | | | | 2.376 |
| Methosulfate | | | | | | | |
| (BTMS) ¹ wt. % | | | | | | | |
| (active) | | | | | | | |
| Cetyl Alcohol | 1.60 | 1.60 | 1.60 | 1.60 | 1.60 | 1.60 | 1.12 |
| (C16 Fatty alcohol)³ | | | | | | | |
| wt. % (active) | | | | | | | |
| Stearyl Alcohol | 2.81 | 2.81 | 2.81 | 2.81 | 2.81 | 2.81 | 2.862 |
| (C18 Fatty Alcohol)⁴ | | | | | | | |
| wt. % (active) | | | | | | | |
| L-Glutamic Acid ⁵ | 0.63 | 0.63 | 0.63 | 0.63 | 0.63 | 0.63 | |
| wt. % (active) | | | | | | | |
| DiSodium EDTA | 0.127 | 0.127 | 0.127 | 0.127 | 0.127 | 0.127 | 0.127 |
| wt. % (active) | | | | | | | |
| Benzyl Alcohol | 0.40 | 0.40 | 0.40 | 0.40 | 0.40 | 0.40 | 0.40 |
| wt. % (active) | | | | | | | |
| Methylchloro- | | | | | | | 0.033 |
| isothiazolinone/ | | | | | | | |
| Methyl ⁷ wt. % | | | | | | | |
| Sodium Benzoate⁶ | 0.25 | 0.25 | 0.25 | 0.25 | 0.25 | 0.25 | |
| wt. % (active) | | | | | | | |
| Perfume | 0.75 | 0.75 | 0.75 | 0.75 | 0.75 | 0.75 | 0.8 |
| Bis-Aminopropyl | 1.2 | 1.2 | 1.2 | 1.2 | 1.2 | 1.2 | |
| Dimethicone | | | | | | | |
| (TAS) ⁸ wt. % | | | | | | | |
| Silicone Quaternium-26 | | | | | | | 3 |
| (PQAS) ¹¹ wt. % | | | | | | | |
| Rice Bran Oil | 1 | 3 | | | | | 1.5 |
| (RBO) wt. % | | | | | | | |
| Caprylic/Capric | | | 1 | 3 | | | 0.5 |
| Triglyceride | | | | | | | |
| (CCT) wt. % | | | | | | | |
| Coconut Oil | | | | | 1 | 3 | |
| (CNO) wt. % | | | | | | | |
| total silicone | 1.2 | 1.2 | 1.2 | 1.2 | 1.2 | 1.2 | 3 |
| (TAS) wt. % | | | | | | | |
| total triglyceride | 1 | 3 | 1 | 3 | 1 | 3 | 2 |
| (RBO + | | | | | | | |
| CCT + CNO) wt. % | | | | | | | |
| ratio of silicone | 1.2:1 | 1.2:3 | 1.2:1 | 1.2:3 | 1.2:1 | 1.2:3 | 3:2 |
| to triglyceride | | | | | | | |
| (RBO) wt. % | | | | | | | |
| Distilled Water | Q.S. | Q.S. | Q.S. | Q.S. | Q.S. | Q.S. | Q.S. |

Adjust pH w Citric acid to pH = 3.5-4.5
¹ Behentrimonium Methosulfate (BTMS) (Incroquat ™ Behenyl TMS-80), 80 wt % active and 20 wt % Isopropyl Alcohol (IPA), available from Croda
²Stearamidopropyl Dimethylamine (SAPDMA) (Gernamin ® SPA,), available from Clariant
³Cetyl alcohol, 95 wt % active level available from Procter & Gamble
⁴Stearyl alcohol, 97 wt % active level, available from Procter & Gamble
⁵ L-Glutamic Acid, available from Ajinomoto
⁶Sodium Benzoate, available from Kalama
⁷ Kathon CG (1.5 wt % active), available from Dow
⁸ Bis-Aminopropyl Dimethicone (TAS) with visc. Of 10,000 cPs at 25° C., available from Momentive Performance Materials
9. Dimethicone (PDMS) (Xiameter ™ PMX-200) Silicone Fluid 10000 cSt, from Dow
10. PEG/PPG-20/23 Dimethicone (SS) (Silsoft ™ 430), available from Momentive Performance Materials
¹¹ Silicone Quaternium-26, available from Momentive Performance Materials

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair conditioning composition comprising:
a L-beta lamellar gel network;
from about 0.1 wt. % to about 10 wt. % silicone;
from about 0.1 wt. % to about 10 wt. % triglyceride;
wherein the ratio of silicone to triglyceride is from about 1:10 to about 10:1;
wherein the composition has a shear stress from about 40 Pa to about 800 Pa @950 1/s according to the Shear Stress Method disclosed herein.

2. The hair conditioning composition of claim 1, wherein the L-beta lamellar gel network comprises surfactant, high melting point fatty compound, and an aqueous carrier.

3. The hair conditioning composition of claim 2, wherein the surfactant is a cationic surfactant.

4. The hair conditioning composition of claim 3, wherein the cationic surfactant is selected from a mono-long alkyl amine; a di-long alkyl quaternized ammonium salt; mono-long alkyl cationic neutralized amino acid esters; a combination of a mono-long alkyl amine and a di-long alkyl quaternized ammonium salt; and a combination of a mono-long alkyl amine and a mono-long alkyl cationic neutralized amino acid esters; and combinations thereof.

5. The hair conditioning composition of claim 1, wherein the high melting point fatty compound is selected from cetyl alcohol, stearyl alcohol, cetearyl alcohol, Brassica alcohol, behenyl alcohol, and combinations thereof.

6. The hair conditioning composition of claim 1, wherein the silicone is selected from polyalkyl siloxanes, polyaryl siloxanes, polyalkylaryl siloxanes, polyether siloxane copolymers, amino substituted silicones, alkylamino substituted silicones, quaternized silicones, aminosilicones, amodimethicones, terminal aminosilicones, branched aminosilicones, silicone polymers containing quaternary groups, and combinations thereof.

7. The hair conditioning composition of claim 1, wherein the silicone is an aminosilicone corresponding to the following formula: 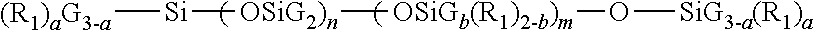
wherein G is selected from the group consisting hydrogen, phenyl, hydroxyl, C₁-C₈ alkyl, and methyl;
a is 0 or an integer having a value from 1 to 3, preferably 1;
b is 0, 1 or 2;
n is a number from 0 to 1,999;
m is an integer from 0 to 1,999; the sum of n and m is a number from 1 to 2,000; and wherein a and m are not both 0;
R₁ is a monovalent radical of formula CqH_{2q}L, wherein q is an integer from 2 to 8; and L is selected from the group consisting of: 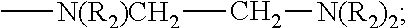
—N(R₂)₂;
—N(R₂)₃A⁻; and
—N(R₂)CH₂—CH₂—NR₂H₂A⁻;
wherein R₂ is selected from the group consisting of hydrogen, phenyl, benzyl, a saturated hydrocarbon radical, and an alkyl radical containing from about C₁ to about C_{20 atoms}; and A⁻ is a halide ion.

8. The hair conditioning composition of claim 7, wherein m is 0, a is 1, q is 3, G is methyl, n is from about 1,500 to about 1,700, and L is —N(CH) or —NH.

9. The hair conditioning composition of claim 7, wherein m is 0, a is 1, q is 3, G is methyl, n is from about 400 to about 600, and L is —N(CH) or —NH.

10. The hair conditioning composition of claim 7, wherein the aminosilicone comprises trimethylsilyamodimethicone.

11. The hair conditioning composition of claim 7, wherein the aminosilicone has a viscosity of from about 1,000 centistokes to about 1,000,000 centistokes.

12. The hair conditioning composition of claim 1, wherein the silicone is silicone polymer corresponding to the following formulas: 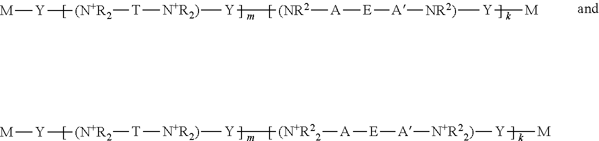
wherein:
m is an average value of from above 0 to 100
k is an average value of from above 0 to 50
M represents a terminal group, comprising terminal ester groups selected from
—OC(O)—Z;
—OS(O)₂—Z;
—OS(O₂)O—Z;
—OP(O)(O—Z)OH;
—OP(O)(O—Z)₂;
wherein Z is selected from monovalent organic residues having up to 40 carbon atoms,
wherein A and A′ each are independently selected from a single bond or a divalent organic group having up to 10 carbon atoms and one or more hetero atoms, and
E is a polyalkylene oxide group of the general formula: 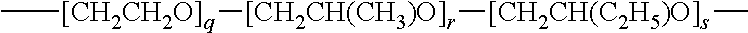
with
q=O to 200,
r=0 to 200,
s=0 to 200,
and q+r+s=1 to 600,
R is selected from monovalent organic groups having up to 22 carbon atoms, and wherein the free valencies at the nitrogen atoms are bound to carbon atoms, R² is selected from hydrogen or R,
Y is a group of the formula: 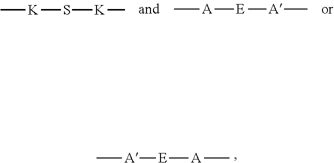
with
S═ 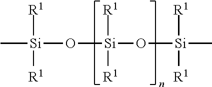
wherein R¹═C₁-C₂₂-alkyl, C₁-C₂₂-fluoroalkyl or aryl,
n=200 to 1000,
K is a bivalent or trivalent straight chain, cyclic and/or branched C₂-C₄₀ hydrocarbon residue, wherein T is selected from a divalent organic group having up to 20 carbon atoms and one or more hetero atoms,
wherein the K residues in the —K—S—K— moiety are identical or different, and are bound to the silicon atom of the residue S via a C—Si— bond.

13. The hair conditioning composition of claim 1, wherein the triglyceride is selected from rice bran oil, camellia oil, rapeseed oil, argon oil, avocado oil, soybean oil, safflower oil, canola oil, hemp seed oil, meadowfoam seed oil, coconut oil, palm kernel oil, caprylic/capric triglyceride, and combinations thereof.

14. The hair conditioning composition of claim 1, wherein the conditioning composition is substantially free of an anionic surfactant.

15. The hair conditioning composition of claim 1, wherein the total surfactant present in the composition is from about 0.1 wt. % to about 20 wt. %.

16. The hair conditioning composition of claim 1, wherein the L-beta lamellar gel network structure may comprise d-spacing of from about 5 nm to about 50 nm according to the Lamella Gel Network Test Method disclosed herein.

17. The hair conditioning composition of claim 2, wherein the composition comprises from about 50 wt. % to about 95 wt. % aqueous carrier.

18. The hair conditioning composition of claim 1, wherein the pH of the composition is less than about 6.

19. A hair conditioning composition comprising:
a L-beta lamellar gel network comprising a cationic surfactant, a high melting point fatty compound, and an aqueous carrier;
from about 0.1 wt. % to about 10 wt. % silicone;
from about 0.1 wt. % to about 10 wt. % triglyceride;
wherein the ratio of silicone to triglyceride is from about 1:5 to about 5:1;
wherein the composition has a shear stress from about 40 Pa to about 800 Pa @950 1/s according to the Shear Stress Method disclosed herein.

20. The hair conditioning composition of claim 19, wherein the silicone is selected from polyalkyl siloxanes, polyaryl siloxanes, polyalkylaryl siloxanes, polyether siloxane copolymers, amino substituted silicones, alkylamino substituted silicones, quaternized silicones, aminosilicones, amodimethicones, terminal aminosilicones, silicone polymers containing quaternary groups, and combinations thereof.
